(19) United States
(12) Patent Application Publication
(10) Pub. No.: US 2006/0177816 A1
(43) Pub. Date: Aug. 10, 2006

(54) Cellular RhoGTPase activation assay

(75) Inventors: Nicole Teusch, Einhausen (DE); Mario Mezler, Deidesheim (DE)

Correspondence Address: WOOD, PHILLIPS, KATZ, CLARK & MORTIMER, 500 W. MADISON STREET, SUITE 3800, CHICAGO, IL, 60661, US

(73) Assignee: Abbott GmbH & Co. KG.

(21) Appl. No.: 11/332,680

(22) Filed: Jan. 13, 2006

Related U.S. Application Data
(60) Provisional application No. 60/644,312, filed on Jan. 14, 2005.

(51) Int. Cl. C12Q 1/00; C12N 5/08

(52) U.S. Cl. 435/4; 435/366

(57) ABSTRACT

The invention relates to a cellular RhoGTPase activation assay based on the determination of changes in the actin cytoskeleton (actin cytoskeletal rearrangement assay); to the recombinant cell lines used to carry out this assay; to the constructs required to produce these cell lines; to the use of these recombinant cell lines for detecting effectors of Nogo receptor (NgR)-dependent signal transduction involving RhoGTPase, in particular for determining effectors of neuronal regeneration; and to assay methods using these recombinant cell lines.

### CROSS REFERENCE TO RELATED APPLICATION

This application claims priority of U.S. provisional patent application Ser. No. 60/644,312 filed on Jan. 14, 2005. The disclosures of which are incorporated herein by reference.

### DESCRIPTION

The invention relates to a cellular RhoGTPase activation assay based on the determination of changes in the actin cytoskeleton (actin cytoskeletal rearrangement (ACR) assay); to the recombinant cell lines used to carry out this assay; to the constructs required to produce these cell lines; to the use of these recombinant cell lines for detecting effectors of Nogo receptor (NgR)-dependent signal transduction involving RhoGTPase, in particular for determining effectors of neuronal regeneration; and to assay methods using these recombinant cell lines.

### PRIOR ART

Rho is a member of the Ras super family of small GTP-binding proteins. Rho, like Ras, also acts as a molecular switch which cycles between an inactive GDP-bound and an active GTP-bound state. These proteins, also called GTPases, bind and hydrolyze guanine nucleotides. Replacement of hydrolyzed GDP by GTP results in a conformational change in the GTPases which permits binding to specific target proteins, called effectors. This mechanism makes it possible for the Rho GTPases to cycle between a biochemically active, GTP-bound and an inactive, GDP-bound conformation. Exchange of GDP for GTP is catalyzed by guanine nucleotide exchange factors (GEFs) since they facilitate the release of GDP and transiently stabilize the nucleotide-free form of GTPase. On the other side, so-called GTPase-activating proteins (GAPs) promote the GTP hydrolysis activity of Rho proteins and thus lead to a rapid conversion into the inactive GDP-bound form. Rho GTPases are, on the basis of this sensitively regulated mechanism, capable of targeted initiation of specific signal responses, in the sense of “molecular switches”, in the presence of extracellular stimuli.

Overall, specific interaction of activated GTPases with various cellular downstream targets makes it possible for a cooperative signal network which allows the control of complex biological processes at various levels to be setup.

To date, fourteen different RhoGTPases have been described (Rac1 to 3; Cdc42; TC10; RhoA to E, G, H and Rnd1,2; cf. also review article by Hall and Bar-Sagi Cell 2000, 103 (2), pp. 227 et seq., which is incorporated herein by reference), the best-characterized family members being Rac1, Cdc42 and RhoA. These GTPases regulate a wide range of cellular functions. The influence of Rac, Rho and Cdc42 on stimulus-dependent changes in the actin cytoskeleton, which forms the basis of many dynamic processes such as, for example, chemotaxis and phagocytosis, represents a major function of Rho GTPases.

For example, activated RhoA induces a signal cascade which leads to significant changes in the cytoskeleton. Thus, for example, the formation of stress fibers, migration or cellular contraction is to be observed in various cell types (cf. Kaibu-chi, K. et al., Annu. Rev. Biochem. (1999), 68, 459486). A hypothetical model of the biochemical mechanism underlying these changes and eventually leading to actin reorganization is described by Narumiya, S. et al., in FEBS Letters, 410 (1997), 68-72.

In the mammalian central nervous system, RhoA appears to play an important role in inhibiting axonal nerve growth and in neuronal growth cone collapse (cf., for example, Lee et al. Nature Reviews, November 2003, Volume 2, 1-7; Schwab, M. E. Current Opinion in Neurobiology (2004), 14, 118-124; Niederöst, B. et al., J. Neurosci. (2002), 22 (23), 10368-10376). The exact cellular signal transmission mechanisms with the aid of which Rho is activated and axonal regeneration in the adult brain is impaired are at present not yet completely understood. However, recent findings indicate that certain components of the myelin sheath such as, for example, Nogo-66 (a fragment comprising 66 amino acids of the NogoA membrane protein), MAG (myelin-associated glycoprotein) or OMgp (oligodendrocyte myelin glycoprotein) inhibit axonal growth by binding to a neuronal protein which is anchored on the axonal membrane and is called Nogo receptor (NgR). Since NgR is bound to the extracellular surface by a lipid anchor, a coreceptor is necessary for coupling to intracellular signal pathways. Recently, the neurotrophin receptor p75, which is known to regulate Rho activation in various tissues, has been identified as possible coreceptor for NgR (Wang, K. C. et al., Nature (2002) 420, 74-78).

Since the adult human brain has only a limited capacity for neuronal regeneration and for structural plasticity (i.e. the ability of one group of nerve fibers to take over the function of another, damaged group of nerve fibers), there is an increasing pharmaceutical interest in blockade of the NgR-induced, Rho-mediated activation of the signal cascade which suppresses this neuronal regeneration. In order to evaluate potential NgR antagonists, it would be desirable to determine RhoGTP (i.e. activated Rho) after stimulation of NgR with myelin proteins such as Nogo-66. However, the crucial prerequisite for such an assay is the generation of cell lines which stably overexpress Rho, because most cells show only a low endogenous expression of this GTPase (cf. N. Teusch, thesis, The function of RhoGTPases in the signal transduction pathways induced by Toll receptors, 2002).

The RBD assay which makes it possible to detect active Rho through affinity precipitation with the Rho-binding domain of the effector protein rhotekin (RBD) is known in the art. This assay is unsuitable for high throughput, because it comprises numerous time-consuming operations such as the preparation of a cell homogenate after activation, precipitation of activated Rho and detection of the precipitated protein. In addition, the results obtainable therewith have low reproducibility and poor quantifiability, making pharmacological characterization of chemical lead structures or antibodies virtually impossible.

It is therefore an object of the present invention to provide an improved assay method for determining effectors of NgR-dependent Rho activation. In particular, it should be possible to carry out the novel assay method more quickly and reliably (reproducibly) and thus make a higher sample throughput possible and, where appropriate, also be standardizable and/or quantifiable.

### BRIEF DESCRIPTION OF THE INVENTION

It has surprisingly been possible to achieve the above object by providing recombinant cell lines which are capable of stable over expression of a functional intracellular factor which is involved in a signal pathway inducing a change in the cytoskeleton of the cell, and additionally show after coexpression of a cellular receptor which interacts with the intracellular factor more reproducible and quantitatively analyzable changes in the actin cytoskeleton of the recombinant cells.

In particular, various cell lines stably expressing RhoGTPase and based on human embryonic kidney cells (e.g. HEK293) have been produced according to the invention.

On this basis, a novel RhoGTPase activation assay (ACR assay) based on NgR-induced changes in the cytoskeleton has been developed.

A surprising observation in this connection was an increased number of recombinant Rho-expressing cells showing changes in the actin cytoskeleton after NgR-dependent stimulation. The production of stably overexpressing Rho cell lines was a prerequisite for this, because most epithelial cells show only low endogenous expression of GTPase.

In contrast to conventional biochemical assays, the ACR assay permits screening and characterization of chemical compounds and/or biomolecules in multiwell plate format, it being possible for these assays to be designed both for high throughput and high content screening. In particular, the use of high content screening systems with image analysis permits the technical disadvantages known in the prior art to be eliminated.

Cell lines which stably overexpress a RhoGTPase in the presence or absence of the nogo receptor and/or of the neurotrophin receptor p75 have not previously been disclosed. In addition, the NgR-induced, Rho-dependent actin cytoskeletal rearrangement such as a cellular contraction and shrinkage had not previously been described in epithelial cells, nor at the implementation of a screening assay based on these observed cellular changes for the purpose of screening and/or of characterization of active substances.

### DESCRIPTION OF THE FIGURES

The invention is now described in detail with reference to the appended figures, which show:

FIG. 1 a model of the Rho-dependent signal cascade which is involved in the inhibition of neuronal growth and which is induced by the NgR/p75 receptor complex. Stimulation of the NgR/p75 receptor complex with myelin proteins such as Nogo-66 activates the GTPase Rho, which binds GTP (guanosine triphosphate). Activated RhoGTPase in turn activates the serine-threonine kinase ROCK (Rho associated kinase), a specific downstream target of Rho. Activation of ROCK leads to phosphorylation of a plurality of cytoskeletal proteins (e.g. myosin light chain kinase (MLCK), CRMP2, cofilin; not shown here), resulting in a change in the cell morphology such as, in particular, a cellular contraction. As is to be expected for a GPI (glycan-phosphatidylinositol)-linked protein, NgR can be released from the cell membrane by treatment with phosphatidylinositol-specific phospholipase C(PI-PLC).

FIG. 2 the principle of the RBD assay for detecting the activation of low molecular weight GTPases by extracellular stimuli. This makes use of the fact that only the GTP-bound form of GTPase (Rho-GTP) interacts with downstream effectors. In the case of Rho, the Rho-binding domain (RBD) of the effector protein rhotekin is used to precipitate active, GTP-bound Rho from cell lysates. GST-RBD beads (polymer beads with RBD anchored via glutathione S-transferase) are incubated with Rho-containing cell lysate and, after centrifugation, RBD-bound Rho-GTP is found in the centrifugate if activated Rho-GTP is present in the lysate. Non-activated Rho (Rho-GDP) by contrast remains in the supernatant.

FIG. 3 the plasmid maps of various plasmids used according to the invention:

pIRES hNgR hp75 (SEQ ID NO: 10) (FIG. 3a)

pcDNA3 hRhoA wt (SEQ ID NO: 15) (FIG. 3b)

pcDNA4 (mycHis)A hRhoA wt (SEQ ID NO: 13) (FIG. 3c)

pcDNA3.1 (V5-His)hp75 No. 16 (SEQ ID NO: 8) (FIG. 3d)

FIG. 4a the immunological detection of expressed Rho by positive clones (#1 to #8), isolated according to the invention, of the triple transfectant HEK293 RhoA/NgR/p75 (clones 1 to 9), positive control (C) obtained by transient transfection with 100 ng of pcDNA4(mycHis)A hRhoA wt; the following was used as marker (M): benchmark prestain protein ladder, invitrogen, order number #10748-010; FIG. 4b the result of a FACS analysis for expressed cell surface receptors hNgR and hp75 of an HEK293 triple transfectant prepared according to the invention (clone #4 and #8) and of untransformed HEK293 cells (top), in each case for analyses with anti-p75 and anti-NgR antibodies.

FIG. 5a the result of an RBD assay with recombinant HEK cell lines of the invention;

FIG. 5b the result of an ACR assay with recombinant HEK cell lines of the invention.

FIG. 6 the results of evaluations of an ACR assay of the invention by various automatic image-analysis methods. Fluorescence micrographs of stimulated and unstimulated HEK293-RhoA/p75/NgR cells are to be seen in FIG. 6 a) of the figure. The relevant bar diagrams which represent the image analysis result are indicated underneath, specifically in FIG. 6b) the average percentage cell length measured with the aid of the Discovery-1 system from Molecular Devices and analysis with the image processing software Metamorph, Molecular Devices; and in FIG. 6c) the number of cytoplasmic vertices (these are a measure of or represent the circumference of the actin cytoskeleton of a cell, but this does not correspond to the cell circumference) determined with the aid of the Atto Pathway HT System from BD Bioimages (Becton Dickinson); FIG. 6d shows representative images illustrating an algorithm detection. Images were taken with the Pathway HT and segmentation was done with Attovision. HEK293-RhoA/NgR/p75 cells were either unstimulated (−) or stimulated with 20 nM AP-Nogo66 (+). The numbers indicate the individual regions of interest (ROIs) detected after segmentation.

FIG. 6e shows the dose response dependency of AP-Nogo66. HEK293-RhoA/NgR/p75 cells were either left unstimulated (−) or were stimulated for 10 min with different concentrations of AP-Nogo66. From a segmentation according to FIG. 6d average cytoplasmic vertices were determined with Attovision and their dependency from the AP-Nogo66 concentration is shown. In a similar way the time course of the actin cytoskeletal rearrangement induced by AP-Nogo66. HEK293-RhoA/NgR/p75 cells were either left unstimulated (−) or were stimulated with 20 nM AP-Nogo66 for different time intervals. The results are depicted in FIG. 6f. FIG. 6g shows the dose response dependency of the ROCK inhibitor Y-27632. HEK293-RhoA/NgR/p75 cells were either left untreated (−) or were pre-treated for 15 min with different concentrations of Y-27632 before stimulation with 20 nM AP-Nogo66. The results as shown are representative of at least three independent experiments.

FIG. 7 Western analysis of AP-Nogo66 with various antisera (anti-AP; anti-Nogo66) of SDS gel electrophoresis. Pro 293 stands for the negative control for blotting and antibody treatment, because only the serum used was loaded here.

FIG. 8 the plasmid map of the plasmid pAP-tag5/PPC/hNOGO66 No. 5 used.

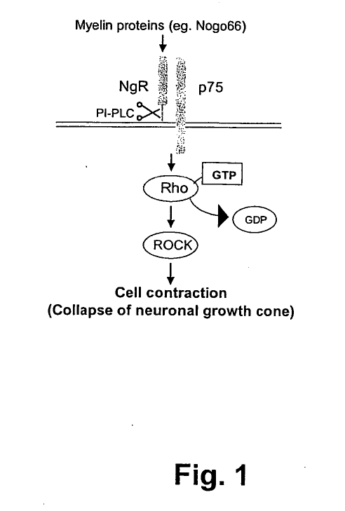

### DETAILED DESCRIPTION OF THE INVENTION

I. Specific Aspects of the Invention

A first aspect of the invention relates to recombinant cell lines which stably express:
a) a functional intracellular factor which is involved in a signal pathway which induces a change in the cytoskeleton of the cell; and/or
b) a membrane-bound cellular receptor which interacts with the intracellular factor.

The invention relates in particular to cell lines which stably express
a) a functional, intracellular RhoGTPase; and/or
b) a membrane-bound receptor which interacts with the RhoGTPase, such as, in particular, a receptor complex comprising:
a functional, membrane-bound neurotrophin receptor p75; and
a functional, membrane-bound Nogo receptor (NgR);

For example, the Rho GTPase in this case is a RhoA GTPase.

There is provision in particular of cell lines where
a) the functional RhoGTPase comprises an amino acid sequence as shown in SEQ ID NO: 16,
b) the functional p75 comprises an amino acid sequence as shown in SEQ ID NO: 9, and
c) the functional NgR comprises an amino acid sequence as shown in SEQ ID NO: 11;
   or cell lines which express functional equivalents of these specific protein factors.

Particularly suitable for producing recombinant cell lines of the invention are those which are capable of reorganization of their actin cytoskeleton induced by binding of a ligand to NgR, where the ligand binding induces for example a Rho activation.

Without being limited thereto, a useful ligand may comprise Nogo-A or an NgR-binding fragment or derivative derived therefrom.

Cell lines of the invention can in particular be derived from an adherent mammalian cell line with extended cell morphology. Examples thereof are human epithelial or fibroblast cell lines.

In a specific embodiment, the cell lines are derived from a human embryonic kidney (HEK) cell line.

A further aspect of the invention relates to the use of a recombinant cell line of the invention for determining effectors of NgR-dependent signal transduction, especially activation of the GTPase Rho.

The invention further relates to assay methods for determining changes in the cytoskeleton of a cell line to be investigated, where a recombinant cell line as defined above or a corresponding primary cell line is cultivated with a ligand which interacts with the cells and which, if appropriate, brings about the change in the cytoskeleton via induction of an intracellular signal pathway, and changes in the reorganization of the cytoskeleton of the cell lines treated in this way are determined. The ligand used in particular in this connection is one which interacts with a membrane-bound receptor.

A “corresponding primary cell line” means in this connection a non-recombinant and otherwise functionally equivalent cell line in which a determinable change in its cytoskeleton can be induced by one of the mechanisms described above.

Examples of primary cells suitable according to the invention are the following cells: cells from the rat dorsal root ganglion (Li & Strittmatter J. Neurosci. 2003 15; 23(10):4219-27) or granular cells from the rat cerebellum (Madura et al. EMBO Rep. 2004; 5(4):412-7).

In a preferred embodiment of this assay method, the cell line is cultivated in the presence or absence of an analyte which is suspected to comprise an effector which modulates the effect of the ligand on the cell. Cultivation with the analyte can in this connection take place simultaneously or sequentially (i.e. earlier or later) in relation to the cultivation with the receptor-binding ligand.

This method is particularly suitable as assay for effectors of NgR-dependent activation of the GTPase Rho, in which case a recombinant cell line as defined above or a corresponding primary cell line is cultivated in the presence of an NgR-binding ligand and in the presence or absence of an analyte which is suspected to comprise the effector, and differences in the reorganization of the cytoskeleton of the cell lines treated in this way are determined.

The assay method of the invention is based in particular on an assay evaluation where the average of at least one of the following parameters is determined in a cell population or one which is representative of one:
a) cell circumference
b) cell length
c) cell width
d) cell area
e) length of the cell projections
f) cell density
g) cytoplasmic area
h) cytoplasm vertices (can be determined after visualization of the cytoskeleton and a measure differing from a) for the cell circumference)
i) refractive index
j) cell-free area of a measured area
k) area covered with cells in a measured area
l) a mathematical linkage of at least two of parameters a) to k), or a parameter which can be derived from at least one of the parameters a) to k), such as, in particular, the ratio of cell length to cell width, or the ratio of cell circumference squared to cell area; or
m) an appropriate reciprocal value of one of the parameters a) to l).

The determination is preferably based on optical methods, if necessary after carrying out a suitable staining of the cells.

The use of automatic image analysis methods is particular advantageous in this connection.

Such assay methods are advantageously used to determine effectors of neuronal regeneration.

It is thus possible for example to assay effectors which partly or completely inhibit Rho activation and/or promote neuronal regeneration.

It is preferred according to the invention in particular to use such assay methods to determine an effector which partly or completely neutralizes a disease- or injury-related inhibition of axonal regeneration and/or a disease- or injury-related collapse of the neuronal growth cone. Such effectors are in particular NgR antagonists.

The cell system of the invention is also used in a screening method, in particular a high-content screening method for effectors, in particular NgR antagonists, of neuronal regeneration. NgR antagonists detectable by means of the present invention may for example be used in the therapy of the following diseases: spinal cord injuries, brain injuries, cerebral cranium trauma, stroke, mechanically caused injuries of the central nervous system, ischemically caused injuries of the central nervous system, injuries of the central nervous system caused by infection, Parkinson's disease, Huntington-Chorea, amytrophous lateral sclerosis and other motoneuronic diseases; Morbus Alzheimer, poliomyelitis, Hirschsprung's disease, paraplegia, diplegia, tetraplegia, infantile neuroaxonal dystrophy (Seitelberger's syndrome), localised neuroaxonal dystrophy (Hallervorden-Spatz syndrome), Wailer degeneration, viral meningitis, bacterial meningitis and axon ischemia, and neurodegenerative diseases, particularly multiple sclerosis, post-polio syndrome, spina bifida, spinal muscular atrophy; tumors, such as brain tumor and spinal cord tumor, and transverse myelitis.

The invention further relates to vectors comprising a nucleic acid sequence coding for functional RhoGTPase or comprising nucleic acid sequences coding for functional p75 and functional NgR.

The invention also relates to methods for producing recombinant cell lines of the invention, where a wild-type cell line of the type described above is transformed with at least one coding nucleic acid sequence such as, for example, one of the vectors described above which codes for a functional RhoGTPase and, if appropriate, for p75 and NgR.

The invention additionally relates to high content screening methods for determining the activation of the GTPase Rho, using an assay method as defined above.

Finally, the invention relates to an apparatus for carrying out such a method, but also assay kits comprising a recombinant cell line as defined above or a corresponding primary cell line, and a ligand for the cellular receptor.

II. Explanation of General Terms

“Functional” protein factors in the context of the invention are those involved in a signal pathway which leads to a change in the cytoskeleton of a eukaryotic cell expressing the factor. Absence or blocking of this functional protein factor would bring about inhibition of the relevant signal pathway.

The terms “expression” or “overexpression” describe in the context of the invention the production or elevation of the intracellular or cell membrane-bound activity of one or more proteins used according to the invention, such as, for example, RhoGTPase, NgR or p75. The degree of overexpression can be controlled for example through the copy number of the genes used or through the strength of the promoter used.

“Stable” expression or overexpression is achieved when the recombinant eukaryotic cell permanently has the ability to express or overexpress the protein.

“Intracellular factors” are those involved as non-membrane-bound, translocatable, soluble factors in the signal pathway which induces a change in the cytoskeleton of the cell.

“Cellular factors” or “cellular receptors” are those which are involved as membrane-bound, optionally non-soluble factors in the signal pathway which induces a change in the cytoskeleton of the cell. They may be bound superficially (i.e. reversibly or irreversibly) to the cell membrane or be anchored via a transmembrane region (i.e. irreversibly). Possible orientations thereof are outward or into the interior of the cell or bidirectional. A cellular factor of the invention should be able on its own or in combination (in a complex) with one or more further optionally membrane-bound factors to interact with an extracellular ligand and, as a consequence thereof, to transmit a binding signal to the intracellular part of the signal pathway which induces a change in the cytoskeleton of the cell.

“Ligands” in the context of the present invention are high or low molecular weight molecules such as, for example, proteins, glycoproteins, nucleic acids or natural or artificially generated fragments thereof which are able to interact, such as, for example, with formation of a non-covalent linkage, with at least one of the cellular receptors or coreceptors which are used according to the invention or are indicated as useful.

“Effectors” in the context of the present invention are in particular those which modulate the receptor-dependent signal transduction which brings about the change in the cytoskeleton of the cells. Effectors may in this connection also be the above “ligands” in particular. “Modulation” means in this connection in particular diminution or complete suppression of the changes in the cell morphology. The effector may in this connection be an agonist, an antagonist, an inverse agonist or a partial agonist. The effector may in this connection be a high or low molecular weight chemical compound which has been isolated from a natural source or synthesized chemically or biochemically. The effector may, however, also be a biomolecule such as, for example, an antibody or an antigen-binding fragment thereof. Further effectors may also be membrane proteins or soluble fragments thereof. Potential effectors are in addition polysaccharides, growth factors, proteoglycans, semaphorins and ephrins.

An “analyte” is a sample to be investigated comprising a natural or artificial mixture of substances or a pure substance. The presence of an effector of the type described above is suspected in the analyte.

“High-content screening” (HCS) stands for a detection method which provides analytical information of great density or complexity. This generally means functional, but in particular cellular screening methods which are carried out with the aid of an automated microscopy system, and analyzed and evaluated with the aid of automated image processing systems/algorithms.

III. Further Information for Implementing the Invention

a) Proteins

Proteins which are used in the context of the invention and are to be mentioned as preferred are:

hNgR (SEQ ID NO: 11) (membrane-bound receptor) (NM_{—}023004)

hp75 (SEQ ID NO: 9 or 12) (membrane-bound coreceptor) (NM_{—}002507)

hRhoAGTPase (SEQ ID NO: 14 or 16) (intracellular factor) (NM_{—}001664)

AP-Nogo66 (receptor ligand) (NogoA: NM_{—}020532, NP_{—}065393)

Alternatives to RhoA which should be mentioned are the GTPases Rac1 to 3 (Brandtlow C Exp Gerontol. 2003; 38(1-2):79-86); Cdc42; TC10; RhoB to E, G, H. (Tanaka et al. EMBO J. 2004 10; 23(5):1075-88)

Alternatives to NgR which should be mentioned are neogenin (receptor for RGM=repulsive guidance molecule), all semaphorin receptors such as, for example, plexin, neuropilin, all ephrin receptors such as, for example, Eph A4 (Rajagopalan et al. Nat Cell Biol. 2004 August; 6(8):756-62), (Moreno-Flores et al. Mol Cell Neurosci. 2002; 20(3):42946). (Hanbali et al. J. Neurochem. 2004; 90(6):1423-31).

Alternatives to p75 which should be mentioned are:
the nerve growth factor receptors TrkA, TrkB, TrkC; (Williams et al. J Biol Chem. 2004 30)
purine receptors able to activate RhoGTPases, such as, for example, P2X7 (Wang et al. Nat Med. 2004; 10(8):821-7)
sortilin (Bronfman et al. EMBO Rep. 2004; 5(9):867-71)
all proteins of the Lingo family such as, for example, Lingo 1, Lingo 2, Lingo 3 (Mi et al. Nat Neurosci. 2004; 7(3):221-8).

Factors which should be mentioned as alternatives to AP-Nogo66 are those able to bind to NgR: Nogo-A, MAG, Omgp and fragments thereof such as NEP1-40; anti-NgR antibodies, anti-p75 antibodies and binding fragments thereof; otherwise tagged Nogo66 such as, for example, GST-Nogo66, His-Nogo66, semaphorins, ephrins, chrondoitin sulfate proteoglycans (CSPGs), RGM, tenascins, nerve growth factors (e.g. NGF, BDNF, NT-3/4, GDNF).

The present invention is, however, not limited to the use of the specifically described protein factors with indicated sequences (to establish a recombinant cell line as basis for carrying out an ACR assay) or ligands (to induce the intracellular signal cascade in the established cell line).

The invention likewise comprises “functional equivalents” of the specifically disclosed proteins/polypeptides.

“Functional equivalents” or analogs of the specifically disclosed polypeptides are in the context of the present invention polypeptides which differ therefrom and which continue to have the desired biological activity such as, for example, receptoror ligand-binding capacity or GTPase activity.

“Functional equivalents” mean according to the invention in particular mutants which have in at least one of the sequence positions of the abovementioned specific sequences an amino acid which differs from that specifically mentioned but, despite this, still have the desired biological activity. “Functional equivalents” thus comprise the mutants obtainable by one or more, such as, for example, 1 to 10, amino acid additions, substitutions, deletions and/or inversions, it being possible for said changes to occur in any sequence position as long as they lead to a mutant having the profile of properties desired according to the invention. Functional equivalence is also present in particular if there is qualitative agreement between mutant and unchanged polypeptide in the activity pattern, i.e. for example identical substrates are converted at a different rate, or identical ligands bind with different strengths. For example, N-terminal Met groups of the abovementioned sequences may be deleted or replaced by another amino acid.

“Functional equivalents” in the above sense are also precursors of the described polypeptides, and functional derivatives and salts of the polypeptides. The term “salts” means both salts of carboxyl groups and acid addition salts of amino groups of the protein molecules of the invention. Salts of carboxyl groups can be prepared in a manner known per se and comprise inorganic salts such as, for example, sodium, calcium, ammonium, iron and zinc salts, and salts with organic bases such as, for example, amines, such as triethanolamine, arginine, lysine, piperidine and the like. Acid addition salts such as, for example, salts with mineral acids such as hydrochloric acid or sulfuric acid and salts with organic acids such as acetic acid and oxalic acid are likewise an aspect of the invention.

“Functional derivatives” of polypeptides of the inventtion can likewise be prepared on functional amino acid side groups or on their N- or C-terminal end with the aid of known techniques. Such derivatives comprise for example aliphatic esters of carboxylic acid groups, amides of carboxylic acid groups, obtainable by reaction with ammonia or with a primary or secondary amine; N-acyl derivatives of free amino groups prepared by reaction with acyl groups; or O-acyl derivatives of free hydroxy groups prepared by reaction with acyl groups.

“Functional equivalents” of course also comprise polypeptides which are obtainable from other organisms, and naturally occurring variants. It is possible for example by sequence comparison to find areas of homologous sequence regions and to ascertain equivalent proteins on the basis of the specific requirements of the invention.

“Functional equivalents” likewise comprise fragments, preferably single domains or sequence motifs, of the polypeptides of the invention, which have for example the desired biological function.

“Functional equivalents” are additionally fusion proteins which have one of the abovementioned polypeptide sequences or functional equivalents derived therefrom and at least one further heterologous sequence, functionally different therefrom, in functional, N- or C-terminal linkage (i.e. with negligible mutual functional impairment of the fusion protein portions). Non-limiting examples of such heterologous sequences are, for example, signal peptides, enzymes, immunoglobulins, surface antigens, receptors or receptor ligands.

“Functional equivalents” also comprised by the invention may have a homology of at least 60%, such as at least 75%, in particular at least 85%, such as, for example, 90%, 95, 96, 97, 98 or 99%, with one of the specifically disclosed sequences, calculated by the algorithm of Pearson and Lipman, Proc. Natl. Acad. Sci. (USA) 85(8), 1988, 2444-2448. A percentage homology of a homologous polypeptide of the invention means in particular percentage identity of the amino acid residues based on the total length of one of the amino acid sequences specifically described herein.

In the event of possible protein glycosylation, functional equivalents of the invention also comprise proteins of the type identified above in deglycosylated or glycosylated form, and modified forms obtainable by altering the glycosylation pattern.

Homologs of the proteins or polypeptides used according to the invention can be generated by mutagenesis, e.g. by point mutation, extension or truncation of the protein.

Homologs of the proteins used according to the invention can be identified by screening combinatorial libraries of mutants such as, for example, truncation mutants. For example, a variegated library of protein variants can be generated by combinatorial mutagenesis at the nucleic acid level, such as, for example, by enzymatic ligation of a mixture of synthetic oligonucleotides. There is a large number of methods which can be used to prepare libraries of potential homologs from a degenerate oligonucleotide sequence. Chemical synthesis of a degenerate gene sequence can be carried out in an automatic DNA synthesizer, and the synthetic gene can then be ligated into a suitable expression vector. Use of a degenerate set of genes makes it possible to provide in one mixture all the sequences which encode the desired set of potential protein sequences. Methods for synthesizing degenerate oligonucleotides are known to the skilled worker (for example Narang, S. A. (1983) Tetrahedron 39:3; Itakura et al. (1984) Annu. Rev. Biochem. 53:323; Itakura et al., (1984) Science 198:1056; Ike et al. (1983) Nucleic Acids Res. 11:477).

b) Nucleic Acids

All the nucleic acid sequences mentioned herein (single- and double-stranded DNA and RNA sequences such as, for example, cDNA and mRNA) can be prepared in a manner known per se by chemical synthesis from the nucleotide units, such as, for example, by fragment condensation of individual overlapping complementary nucleic acid units of the double helix. Chemical synthesis of oligonucleotides is possible for example in a known manner by the phosphoamidite method (Voet, Voet, 2nd edition, Wiley Press New York, pages 896-897). The addition of synthetic oligonucleotides and filling in of gaps with the aid of the Klenow fragment of DNA polymerase and ligation reactions, and general cloning methods are described in Sambrook et al. (1989), Molecular Cloning: A laboratory manual, Cold Spring Harbor Laboratory Press.

The nucleic acid molecules of the invention may additionally comprise untranslated sequences from the 3′ and/or 5′ end of the coding gene region.

The term “isolated nucleic acid sequence” stands for a gene sequence in a state which does not occur naturally. In general, this means that it has been isolated from its natural state or has been synthesized or prepared in an environment which does not occur naturally. In particular, this comprises nucleic acid molecules which have been formed or obtained in vitro; including genomic DNA fragments, recombinant or synthetic molecules, and nucleic acids in combination with heterologous nucleic acids. The term also extends to the genomic DNA, cDNA, RNA or parts thereof. The nucleic acid sequences considered herein also comprise oligonucleotides suitable as genetic probes and suitable for example for identifying coding sequences of functionally equivalent protein factors which can be used according to the invention.

An “isolated” nucleic acid molecule is separated from other nucleic acid molecules which are present in the natural source of the nucleic acid, and may in addition be substantially free of other cellular material or culture medium if it is prepared by recombinant techniques, or free of chemical precursors or other chemicals if it is synthesized chemically.

The term “gene” or “gene sequences” is used herein in its most general meaning and comprises all consecutive sequences of nucleotide bases which code, directly or via a complementary series of bases, for the amino acid sequence of a protein which can be used according to the invention, in particular RhoGTPases and cellular receptors interacting therewith. Such amino acid sequences may be a full-length protein or an active truncated form thereof, or they may correspond to a particular region, such as an N-terminal, C-terminal or an internal region of the protein.

A nucleic acid molecule of the invention can be isolated by means of standard techniques of molecular biology and the sequence information provided according to the invention. For example, cDNA can be isolated from a suitable cDNA library by using one of the specifically disclosed complete sequences or a segment thereof as hybridization probe and standard hybridization techniques (as described for example in Sambrook, J., Fritsch, E. F. and Maniatis, T. Molecular Cloning: A laboratory manual, 2nd edition, Cold Spring Harbor Laboratory, Cold Spring Harbor Laboratory Press, Cold Spring Harbor, N.Y., 1989). In addition, a nucleic acid molecule comprising one of the disclosed sequences or a segment thereof can be isolated by polymerase chain reaction, using the oligonucleotide primers constructed on the basis of this sequence. The nucleic acid amplified in this way can be cloned into a suitable vector and be characterized by DNA sequence analysis. The oligonucleotides used according to the invention may moreover be prepared by standard synthetic methods, e.g. using an automatic DNA synthesizer.

The invention further comprises the nucleic acid molecules complementary to the specifically described nucleotide sequences, or a segment thereof.

The nucleotide sequences disclosed according to the invention make it possible to generate probes and primers which can be used to identify and/or clone homologous sequences in other cell types and organisms. Such probes and primers usually comprise a nucleotide sequence region which hybridizes under stringent conditions to at least about 12, preferably at least about 25 such as, for example, about 40, 50 or 75 consecutive nucleotides of a sense strand of a nucleic acid sequence of the invention or of a corresponding antisense strand.

Further nucleic acid sequences which can be used according to the invention are derived from the sequences described herein and differ therefrom through addition, substitution, insertion or deletion of single or multiple nucleotides, but continue to code for polypeptides having the desired profile of properties.

The invention also comprises those nucleic acid sequences which comprise so-called silent mutations or are modified, by comparison with a specifically mentioned sequence, in accordance with the codon usage of a specific source or host organism, as well as naturally occurring variants such as, for example, splice variants or allelic variants thereof. Sequences obtainable by conservative nucleotide substitutions (i.e. the relevant amino acid is replaced by an amino acid of the same charge, size, polarity and/or solubility) are likewise an aspect.

The invention also relates to molecules derived from the specifically disclosed nucleic acids by sequence polymorphisms. These genetic polymorphisms may exist between individuals within a population owing to natural variation. These natural variations normally bring about a variance of from 1 to 5% in the nucleotide sequence of a gene.

The invention further also comprises nucleic acid sequences which hybridize with the abovementioned coding sequences or are complementary thereto. These polynucleotides can be found by screening genomic or cDNA libraries and, if appropriate, amplification therefrom with suitable primers by means of PCR and subsequent isolation with suitable probes for example. A further possibility is to transform suitable microorganisms with polynucleotides or vectors of the invention, multiply the microorganisms and thus the polynucleotides, and subsequently isolate them. An additional possibility is to synthesize polynucleotides of the invention by a chemical route.

The property of being able to “hybridize” onto polynucleotides means the ability of a polynucleotide or oligonucleotide to bind under stringent conditions to an almost complementary sequence, while there are nonspecific bindings between noncomplementary partners under these conditions. For this purpose, the sequences should be 70-100%, preferably 90-100%, complementary. The property of complementary sequences being able to bind specifically to one another is made use of, for example, in the Northern or Southern blotting technique or in the primer binding in PCR or RT-PCR. Oligonucleotides with a length of 30 base pairs or more are normally employed for this purpose. Stringent conditions mean, for example, in the Northern blotting technique the use of a washing solution at 50-70° C., preferably 60-65° C., for example 0.1×SSC buffer with 0.1% SDS (20×SSC: 3M NaCl, 0.3M Na citrate, pH 7.0) for eluting nonspecifically hybridized cDNA probes or oligonucleotides. In this case, as mentioned above, only nucleic acids with a high degree of complementarity remain bound to one another. The setting up of stringent conditions is known to the skilled worker and is described for example in Ausubel et al., Current Protocols in Molecular Biology, John Wiley & Sons, N.Y. (1989), 6.3.1-6.3.6.

C) Expression Constructs and Vectors:

The invention additionally relates to expression constructs comprising a nucleic acid sequence coding for a polypeptide used according to the invention, under the genetic control of regulatory nucleic acid sequences; and vectors comprising at least one of these expression constructs. Such constructs of the invention preferably comprise a promoter 5′-upstream from the particular coding sequence, and a terminator sequence 3′-downstream and, if appropriate, other usual regulatory elements in particular each operatively linked to the coding sequence. “Operative linkage” means the sequential arrangement of promoter, coding sequence, terminator and, if appropriate, further regulatory elements in such a way that each of the regulatory elements is able to comply with its function as intended for expression of the coding sequence. Examples of operatively linkable sequences are targeting sequences, and enhancers, polyadenylation signals and the like. Further regulatory elements comprise selectable markers such as resistance genes, amplification signals, origins of replication and the like. Suitable regulatory sequences are described for example in Goeddel, Gene Expression Technology: Methods in Enzymology 185, Academic Press, San Diego, Calif. (1990).

In addition to the artificial regulatory sequences it is possible for the natural regulatory sequence still to be present in front of the actual structural gene. This natural regulation can be switched off if appropriate by genetic modification, and the expression of the genes can be increased or decreased. The gene construct may, however, also have a simpler structure, that is to say no additional regulatory signals are inserted in front of the structural gene, and the natural promoter with its regulation is not deleted. Instead the natural regulatory sequence is mutated so that regulation no longer takes place, and gene expression is enhanced or diminished. The nucleic acid sequences may be present in one or more copies in the gene construct.

Examples of useful promoters are: cos, tac, trp, tet, trp-tet, Ipp, lac, Ipp-lac, lacIq, T7, T5, T3, gal, trc, ara, SP6, lambda-PR or in the lambda-PL promoter, which are advantageously used in gram-negative bacteria; and the gram-positive promoters amy and SPO2, the yeast promoters ADC1, MFalpha, AC, P-60, CYC1, GAPDH or the plant promoters CaMV/³⁵S, SSU, OCS, lib4, usp, STLS1, B33, not or the ubiquitin or phaseolin promoter. Mention should also be made of inducible promoters such as, for example, light- and, in particular, temperature-inducible promoters such as the PᵣPₗ promoter. It is possible in principle for all natural promoters with their regulatory sequences to be used. In addition, it is also possible advantageously to use synthetic promoters.

The regulatory sequences or factors are able in this connection preferably to influence positively, and thus increase or decrease, the expression. Thus, an enhancement of the regulatory elements may take place advantageously at the transcription level by using strong transcription signals such as promoters and/or enhancers. However, it is also possible in addition to enhance translation by, for example, improving the stability of the mRNA.

An expression cassette is prepared by fusing a suitable promoter to a suitable coding nucleotide sequence and to a terminator or polyadenylation signal. Conventional techniques of recombination and cloning are used for this purpose, as described for example in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Harbor Laboratory, Cold Spring Harbor, N.Y. (1984) and in Ausubel, F. M. et al., Current Protocols in Molecular Biology, Greene Publishing Assoc. and Wiley Interscience (1987).

For expression in a suitable host organism, the recombinant nucleic acid construct or gene construct is advantageously inserted into a vector which makes optimal expression of the genes in the host possible. Vectors are well known to the skilled worker and can be found, for example, in “Cloning Vectors” (Pouwels P. H. et al., editors, Elsevier, Amsterdam-New York-Oxford, 1985). Vectors mean not only plasmids but also all other vectors known to the skilled worker, such as, for example, phages, viruses such as SV40, CMV, baculovirus and adenovirus, transposons, IS elements, phasmids, cosmids and linear or circular DNA. These vectors may undergo autonomous replication in the host organism or chromosomal replication.

Examples of suitable expression vectors which may be mentioned are:

Conventional fusion expression vectors such as pGEX (Pharmacia Biotech Inc; Smith, D. B. and Johnson, K. S. (1988) Gene 67:31-40), pMAL (New England Biolabs, Beverly, Mass.) and pRIT 5 (Pharmacia, Piscataway, N.J.), with which respectively glutathione S-transferase (GST), maltose E-binding protein and protein A are fused to the recombinant target protein.

Non-fusion protein expression vectors such as pTrc (Amann et al., (1988) Gene 69:301-315) and pET 11d (Studier et al. Gene Expression Technology: Methods in Enzymology 185, Academic Press, San Diego, Calif. (1990) 60-89).

Yeast expression vector for expression in the yeast S. cerevisiae, such as pYep-Sec1 (Baldari et al., (1987) Embo J. 6:229-234), pMFa (Kurjan und Herskowitz (1982) Cell 30:933-943), pJRY88 (Schultz et al. (1987) Gene 54:113-123) and pYES2 (Invitrogen Corporation, San Diego, Calif.). Vectors and processes for constructing vectors suitable for use in other fungi, such as filamentous fungi, comprise those which are described in detail in: van den Hondel, C. A. M. J. J. & Punt, P. J. (1991) “Gene transfer systems and vector development for filamentous fungi”, in: Applied Molecular Genetics of Fungi, J. F. Peberdy et al., Eds., p. 1-28, Cambridge University Press: Cambridge.

Baculovirus vectors which are available for expression of proteins in cultured insect cells (for example Sf9 cells) comprise the pAc series (Smith et al., (1983) Mol. Cell Biol. 3:2156-2165) and the pVL series (Lucklow and Summers (1989) Virology 170:31-39).

Plant expression vectors such as those described in detail in: Becker, D., Kemper, E., Schell, J. and Masterson, R. (1992) “New plant binary vectors with selectable markers located proximal to the left border”, Plant Mol. Biol. 20:1195-1197; and Bevan, M. W. (1984) “Binary Agrobacterium vectors for plant transformation”, Nucl. Acids Res. 12:8711-8721.

Mammalian expression vectors such as pCDM8 (Seed, B. (1987) Nature 329:840) and pMT2PC (Kaufman et al. (1987) EMBO J. 6:187-195).

Further suitable expression systems for prokaryotic and eukaryotic cells are described in chapters 16 and 17 of Sambrook, J., Fritsch, E. F. and Maniatis, T., Molecular Cloning: A Laboratory Manual, 2nd edition, Cold Spring Harbor Laboratory, Cold Spring Harbor Laboratory Press, Cold Spring Harbor, N.Y., 1989.

d) Recombinant Hosts:

The vectors of the invention can be used to produce recombinant microorganisms/cell lines which are transformed, for example, with at least one vector of the invention.

The recombinant constructs of the invention described above are advantageously introduced into a suitable host system and expressed. Cloning and transfection methods familiar to the skilled worker, such as, for example, coprecipitation, protoplast fusion, electroporation, retroviral transfection and the like, are preferably used to bring about expression of said nucleic acids in the particular expression system. Suitable systems are described, for example, in Current Protocols in Molecular Biology, F. Ausubel et al., Eds., Wiley Interscience, New York 1997, or Sambrook et al. Molecular Cloning: A Laboratory Manual, 2nd edition, Cold Spring Harbor Laboratory, Cold Spring Harbor Laboratory Press, Cold Spring Harbor, N.Y., 1989.

Suitable host organisms are in principle all organisms which enable expression of the nucleic acids of the invention, their allelic variants, their functional equivalents or derivatives.

The cell lines suitable for establishing the ACR assay system of the invention are those in which a microscopically observable reorganization of the actin cytoskeleton is inducible. Such cell lines are derived in particular from an adherent mammalian cell line with extended cell morphology, such as, in particular, a preferably human, epithelial or fibroblast cell line. However, it is also possible to use mammalian cell lines of non-human origin such as, for example, mouse, rat, monkey, goat, shaft, bovine, hamster cell lines and the like. Examples which may be mentioned are: COS1, COS7, SWISS or NIH 3T3, CHO, PC12. Further examples which should be mentioned are primary cells (e.g. cortical neurons) for which a nerve outgrowth in connection with Rho has been described many times (cf., for example, Dergham et al. 2003, J. Neuroscience 22(15), p. 6570).

Human embryonic kidney cell lines are particularly suitable. Examples which may be mentioned are in particular HEK293 cells. In addition, all adherent epithelial and fibroblast cell lines, including cancer cell lines such as, for example, the neuroglioma cell line U87MG (Liao et al. 2004, J. Neurochem. 90(5), p. 1156), SH-SY5Y (=neuroblastoma), HeLa, CACO-2, are suitable in principle. Selection of successfully transformed organisms can take place by marker genes which are likewise present in the vector or in the expression cassette. Examples of such marker genes are genes for antibiotic resistance and for enzymes which catalyze a color-forming reaction which causes staining of the transformed cell. These can then be selected by automatic cell sorting. Microorganisms which have been successfully transformed with a vector and harbor an appropriate antibiotic resistance gene (e.g. G418 or hygromycin) can be selected by appropriate antibiotic-containing media or nutrient media. Marker proteins presented on the cell surface can be used for selection by means of affinity chromatography.

e) Procedure for the Assay of the Invention

The detection method of the invention can be employed as a single investigation or as part of large-scale screenings. However, the procedure always follows substantially the same scheme.

A defined number (e.g. 10² to 10⁶) of recombinant, preferably freshly cultivated cells is introduced into a suitable assay vessel, e.g. well of a microtiter plate, in culture medium. The cells are then incubated with an effective amount of receptor-binding ligands (for AP-Nogo66 e.g. 1 to 50, ng/ml such as, in particular, about 10 ng/ml), such as, for example, for 1 to 30 minutes, at a suitable temperature, such as, for example, in the range of about 20 to 40° C. Optimal assay conditions can be established directly by a skilled worker in a few preliminary tests.

Stimulation takes place in the presence or absence of an analyte being investigated, which comprises an effector of the ligand-receptor interaction. Various concentrations of the analyte are usually employed. Addition of the analyte can moreover take place before or after the time of addition of the ligands or at the same time.

For the evaluation, the stimulated cells are stained and visualized, preferably after immobilization on a support such as, for example, the microtiter plate used. Suitable methods for immobilization, staining and visualization of the cells are known to the skilled worker (cf., for example, Zell-und Gewebekultur, 4th edition, Sektrum akademischer Verlag, Fischer).

The changes in the cytoskeleton are usually so significant that a qualitative statement of the assay result can be made simply by comparison with a visualized reference sample.

Quantitative statements can be obtained by using automatic image analysis systems which determine measured parameters suitable for the investigated cell population and compare between the individual samples. Examples of such parameters are cell circumference, cell length, cell width, cell area, length of the cell projections, a mathematical linkage of at least two of these parameters or a corresponding reciprocal of one such parameter.

Screening methods can moreover be automated in relation to sample preparation, procedure and evaluation of the measurement, so that the time taken per single analysis can be significantly reduced. Suitable apparatuses are available from various manufacturers such as, for example, from Becton Dickinson, Molecular Devices, Cellomics, Amersham Biosciences.

The present invention is now described in more detail with reference to the following non-limiting exemplary embodiments.

### EXPERIMENTAL SECTION

I. General Information

a) Constituents of the Culture Media

RPMI-Glutamax (Invitrogen, Carlsbad, USA)

Geneticin (G418); (antibiotic; selection marker) (Invitrogen)

Fetal Bovine Serum (FBS) Dialyzed, (Invitrogen)

Antibiotic-Antimycotic (Invitrogen)

Zeocin (antibiotic; selection marker) (Invitrogen)

LIPOFECTAMINE (Invitrogen)

b) Cells

Human embryonic kidney cells (HEK293) (ATCC=American Tissue Culture Collection, order No.: CRL-1573) were stably transfected with Rho (HEK293-RhoA) or with Rho in combination with the receptor complex NgR/p75 (HEK293-RhoA/NgR/p75). The cells were maintained in RPMI-Glutamax medium supplemented with 5% heat-inactivated fetal bovine serum (FBS), HEPES (10 mM), penicillin (100 U/ml), streptomycin (100 mM) and either neomycin (HEK293-RhoA) or neomycin in combination with zeocin (HEK293-RhoA/NgR/p75).

c) Reagents

| | |
|---|---|
| Nogo-66, comprising an N-terminal | |
| alkaline phosphatase Tag (AP-Nogo-66) | |
| L-alpha-lysophosphatitic acid (LPA) | Sigma, Munich, DE |
| Phosphatidylinositol-specific | Sigma |
| phospholipase C (PI-PLC) | |
| Beads coated with Rho binding domain | Upstate Biotechnology, |
| (RBD) of rhotekin (amino acids 7-89) | Lake Placid, USA |
| Anti-RhoA antibodies | Santa Cruz Biotechnology, |
| | Santa Cruz, USA |
| Anti-Nogo66 antibodies (NogoA12-A) | Alpha Diagnostics |
| | International, San Antonio, |
| | Texas, USA |
| Alexa Phalloidin 568 or 488 (dye for | Molecular Probes, Eugene, |
| fluorescent staining of F-actin) | USA |
| ECL (Western Blotting Detection Reagent, | Pierce, Rockford, USA |
| electrochemiluminescence), | |
| DAPI (Hoechst 33342, trihydrochloride, | Molecular Probes Inc., |
| trihydrate) | Eugene, USA |

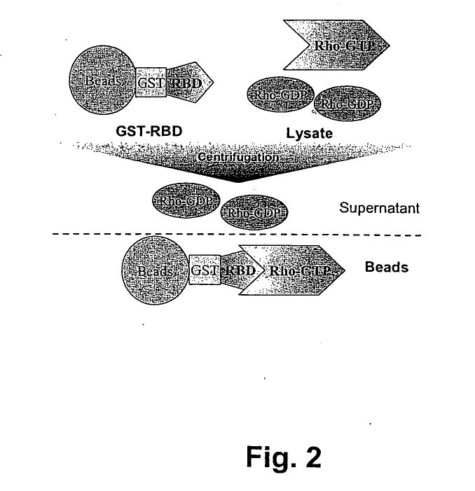

II. General Assay Methods
a) RBD Assay (cf. also FIG. 2)

The HEK cells either remained unstimulated or were stimulated (e.g. with LPA or AP-Nogo-66) for a period of from 5 to 10 minutes after a 2-hour starvation period (by incubation with serum-free RPMI-Glutamax). HEK cells (1×10⁶ per well) were cultured in 6-well plates (volume of mixture 1000 μl/well). Before the stimulation, a particular volume appropriate for the concentration of the AP-Nogo66 batch employed (e.g. 100 μl of medium was removed from each well and the volume was correspondingly made up again to 1000 μl with AP-Nogo66, to result in a defined final concentration of AP-Nogo66 on the cells. Following the stimulation, the AP-Nogo66 medium solution was completely removed by aspiration. The cells were then washed with ice-cold PBS (phosphate-buffered saline) and lyzed in 50 mM Tris, pH 7.5 (comprising NaCl (500 mM), EDTA (1 mM), MgCl₂ (10 nM), 10% glycerol, NaF (5 mM), DTT (1 mM), 1% Nonidet P-40 in combination with the protease inhibitors PMSF (1 mM), leupeptin (1 μg/ml) and aprotinin (1 μg/ml)).

Cell lysates (e.g. from HEK293-RhoA and HEK293-RhoA/NgR/p75; 400 μg of total protein in each case) were incubated with 40 μg of the GST-RBD beads (comprising recombinant fusion protein composed of glutathione S-transferase and RBD with amino acids 7 to 89 of human rhotekin) at 4° C. for 1 hour. After the beads had been washed 3 times with lysis buffer, the bound protein (GTP-bound, biochemically active Rho) was analyzed by SDS-PAGE and immunoblotting. The immunoblotting was carried out with anti-Rho antibody and the visualization took place by incubation with a horseradish peroxidase-conjugated secondary antibody and chemiluminescence enhancer (ECL).

b) ACR Assay with Immunofluorescence Measurement:

1×10⁴ cells were transferred into microtiter plates with 96 wells two days before the stimulation. The cells were stimulated with a suitable reagent (e.g. LPA or AP-Nogo-66) in culture medium (RPMI-Glutamax) comprising 5% FCS. After a stimulation period of 5 to 10 minutes, the activation was stopped with cold PBS. The cells were fixed with 3-4% strength paraformaldehyde solution, permeabilized with PBS comprising 0.2% Triton X-100, and incubated with phalloidin Alexa 568 or 488 for 30-45 minutes. Incubation with DAPI additionally took place for 5 minutes for nuclear staining. The cells were visualized with the aid of an epifluorescence microscope (Axiovert 25). Fluorescence micrograms were recorded using a cooled Zeiss CCD camera.

c) FACS Analysis

Reagents:

| | |
|---|---|
| Mouse anti-human p75; IgG monoclonal antibody | Sigma N-5408 |
| Anti-mouse-FITC | Sigma F-5262 |
| Goat anti-human NogoR | R&D Systems; |
| | AF1208 |
| Anti-goat-FITC | Sigma F7367 |

Procedure:

Firstly, the HEK293 cells were washed with PBS. Adherent cells were detached with PBS comprising 5 mM EDTA. Subsequently, 2×10⁶ cells were transferred into an Eppendorff vessel and centrifuged at 1300 rpm for 2 minutes. The cells were resuspended in PBS comprising 1% BSA (1 ml/vessel) and centrifuged at 1300 rpm for 2 minutes.

The pellets were again resuspended with 0.1 ml of PBS/1% BSA including primary antibody (mouse anti-human p75 1:100, or goat anti-human NogoR 1:50) and incubated at 4° C. for 90 minutes. Subsequently, 0.1 ml of PBS/1% BSA was added, mixed and centrifuged at 1300 rpm for 2 minutes.

The pellets were resuspended in PBS comprising 1% BSA (0.1 ml/vessel) and secondary antibody (anti-mouse-FITC 1:100; or anti-goat-FITC 1:100) and incubated at 4° C. for 60 minutes.

PBS comprising 1% BSA (1 ml/vessel) was added, mixed and centrifuged at 1300 rpm for 2 minutes. After centrifugation, the pellets were again resuspended in PBS/0.1% propidium iodide to detect dead cells. Resuspension was followed by FACS analysis (FACScan, Becton Dickinson, Heidelberg, Germany).

III. Exemplary Embodiments

### Example 1

### Preparation of AP-Nogo66:

a) Cloning of the Nogo66 fragment of hNogoA

The starting point was the published hNogoA sequence (NCBI): AF148537. The two following synthetic oligonucleotides were derived from the published sequence:
Mez 402: GCCACCATGAGGATATACMGGGTGTGATCC (SEQ ID NO: 1); oligo from amino acid R1055 (italic) with start ATG (underlined) and Kozak sequence (bold)
Mez 404: CTTCAGAGMTCMCTAAATCATC (SEQ ID NO: 2); oligo up to amino acid K1120 (bold)

A PCR was carried out with these two oligos in frontal cortex cDNA as template (prepared using Superscript first strand synthesis system for RT-PCR; Invitrogen, Carlsbad, Calif.) (Novak et al., Brain Res. Mol. Brain, 2002, 107(2): 183-189). The reaction was carried out by a standard method, like Innis et al. (PCR Protocols. A Guide to Methods and Applications, Academic Press (1990)) with Herculase (Stratagene, La Jolla, USA). The resulting DNA fragment with a size of 207 bp was purified using the QIAEX II gel extraction kit (QIAGEN GmbH, Hilden, Germany) as specified by the manufacturer. The amplified, purified fragment was put into pcDNA3.1V5-His TOPO (SEQ ID NO: 6) (pcDNA3.1N5-His TOPO TA Expression Kit, #K4800-01). The construct obtained in this way, pcDNA3.1V5-His hNOGO66, was used to transform E. coli TOP10 cells (Invitrogen, Carlsbad, Calif.) by standard methods as described in Sambrook et al. (Molecular Cloning. A Laboratory Manual, Cold Spring Harbor, (1989)). Selection of plasmid-harboring cells was achieved by the antibiotic ampicillin.

Identification of nine clones by PCR showed a band of the correct size of 207 bp for three clones. The correctness of the sequence was checked by sequence analysis.

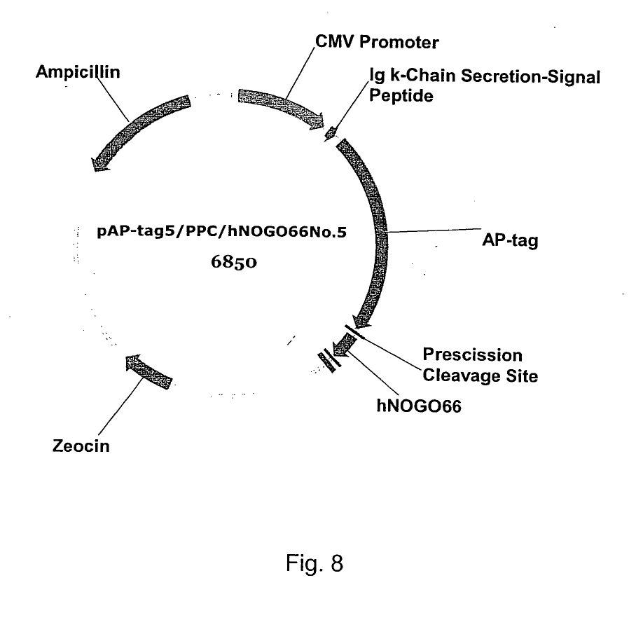

The coding Nogo66 sequence was cut out of the above plasmid pcDNA3.1V5-His hNOGO66 with XbaI and cloned into the plasmid pAP-tag5 (GenHunter Cooperation, Nashville, Tenn., USA) via XbaI. The plasmid pAP-tag5/PPC/hNOGO66 No. 5 (SEQ ID NO: 7), depicted in FIG. 8, is obtained.

b) Preparation of the Stable Cell Line:

HEK293 cells were cultured in growth medium (DMEM with 10% fetal bovine serum with addition of penicillin/streptomycin) at 37° C. and 5% CO₂. For the transfection, the cells were seeded in plates with 6 wells (1×10⁶ per well) and incubated overnight. The cells were transfected with a cocktail which comprised 1 μg of plasmid DNA (pAP-tag5/PPC/hNOGO66 No. 5) and 3 μl of Fugene6 (ROCHE Diagnostics, Mannheim) as stated by the manufacturer. Two days later, the cells were detached by treatment with trypsin and transferred into a cell culture bottle with a base area of 175 cm², and selection was started by adding 150 μg/ml zeocin to growth medium. Zeocin-resistant cell colonies which grew were detached with trypsin after about 4 weeks and isolated in 96-well plates using a cell sorter. After about three weeks, an aliquot of the medium supernatant of the grown single-cell colonies, whose confluence at this time was estimated at between 5 and 85%, was pipetted onto nitrocellulose. Protein expression was detected by adding a solution of NBT (nitroblue tetrazolium salt) and BCIP (5-bromo-4-chloro-3-indolyl phosphate) via the activity of the alkaline phosphatase on the basis of the resulting stain.

c) Expression of AP-Nogo66:

An HEK293 clone (clone No. 5) which produces AP-Nogo66 was expanded in culture medium (RPMI Glutamax+10% FCS, 150 μg/ml zeocin, antibiotic/antimycotic) by multiple passage (about 20) in culture bottles. The AP-Nogo66 expression was checked after every second or third passage in a dot blot assay (with NBT/BCIP reagent).

In order to produce AP-Nogo66 in serum-free medium, the cells were initially cultured almost to confluence in triple flasks, then the medium was changed to expression medium (Pro293a —CDM medium (Biowhittaker; #12-764Q), 2 mM glutamine, antibiotic/antimycotic). These cells were then cultured at 37° C. for 34 days, and the supernatants were removed and centrifuged (1500 rpm, 5 min). The supernatants were collected at −80° C. For further processing, they were thawed, mixed with proteinase inhibitors (PMSF 0.1 mM, Pefabloc SC 1 mM) and concentrated in Amicon tubes (Millipore), and the AP-Nogo66 concentration was determined (about 3 μg/ml) (Mw of monomeric AP: 67 kDa and monomeric AP-Nogo66 about 75 kDa in SDS gels).

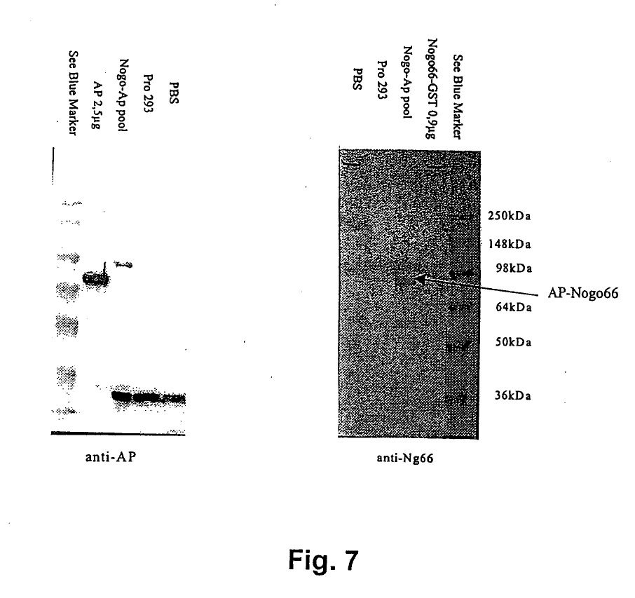

The AP-Nogo66 pool was immunoprecipitated with anti-AP-agarose. The precipitate was denatured by heating in the presence of 5% mercaptoethanol at 95° C. for 10 minutes, and thus removed from the agarose beads, and finally verified by Western analysis using antibodies with specificity for AP and Nogo66. An SDS gel is depicted in FIG. 7.

It was determined by gel chromatography (Superose 12, Amersham Biosciences, mobile phase: 20 mM sodium phosphate, 140 mM sodium chloride, pH 7.4) that AP-Nogo66 migrates as dimer and has an Mw of about 140 kDa.

It was found in preliminary experiments that AP-Nogo66, but not GST-Nogo66, was functionally active (experiments not shown). It is therefore presumed that NgR ligands must possibly all be in the form of dimers in order to be functional. As shown by the crystal structure of the alkaline phosphatase, the dimerization is induced by the AP tag.

### Example 2

### Preparation of Constructs to Generate Recombinant HEK Cell Lines

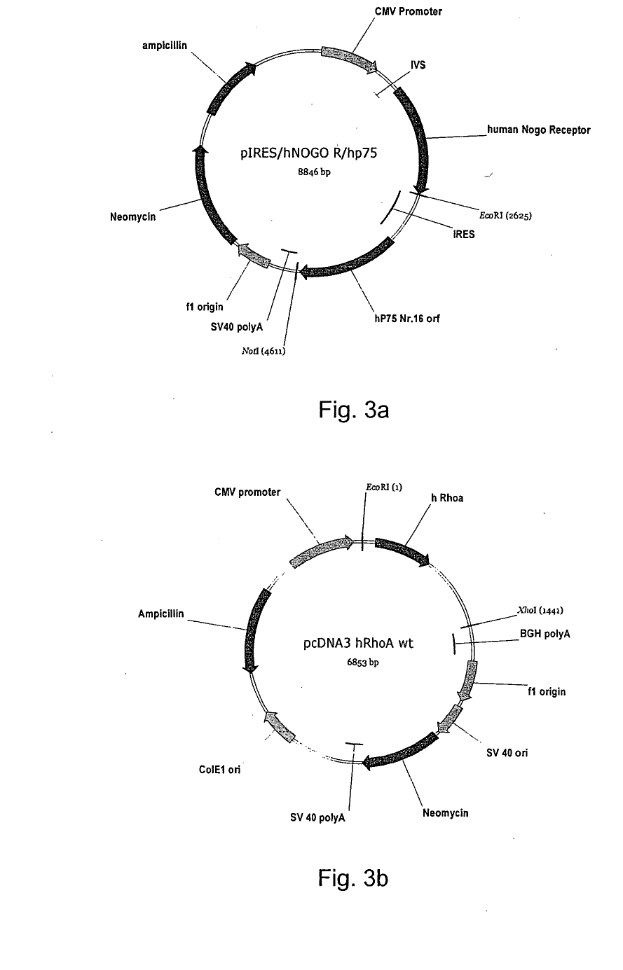

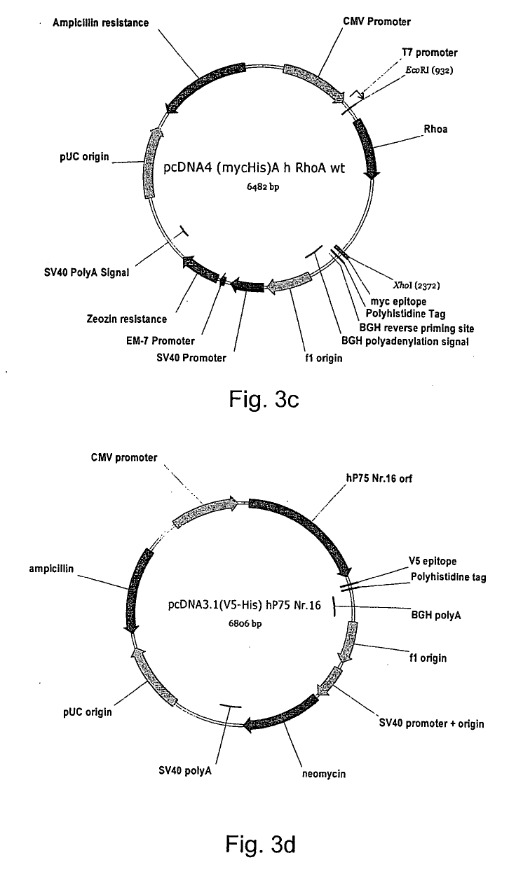

a) Cloning of hp75; Preparation of pcDNA3.1(V5-His)hp75 No. 16 (FIG. 3d)

The starting point was the published sequences for human p75: NM_{—}002507; M14764 (which are both 100% identical). The following oligonucleotides were derived from the published sequence:

Mey 36: GCCACCATGGGGGCAGGTGCCACC (SEQ ID NO: 4); oligo with start codon (bold) with Kozak sequence (italic)

Mey 35: TCACACTGGGGATGTGGCAG (SEQ ID NO: 3); oligo with stop codon (bold) in a base exchange of T instead of C (underlined) because the oligo is derived from the published rat sequence

Mey 71: GCAGCCCCATCAGTCCGC (SEQ ID NO: 5); oligo starting 64 base pairs in front of ATG

A PCR was then carried out (in analogy to the Nogo 66 cloning, example 1) with the primer pair Mey 35/71 in cDNA from the cell line SH-SY5Y (human neuroblastoma cell line ATCC #CRL-2266). The PCR with Mey 35/71 afforded a 1348 bp fragment. This was purified.

A subsequent PCR with the primer pair Mey 35/36 in the Mey 35/71 fragment yielded a clear band (fragment size: 1284 bp). This was purified. This band was then inserted into pcDNA3.1 N5-His TOPO vector (pcDNA3.1 (V5-His) TOPO TA Expression Kit Invitrogen #K4800-01) uncut (topoisomerase principle). The construct obtained in this way (pcDNA3.1 hp75) was used to transform TOP10 cells by standard methods (analogous to Nogo 66 cloning, example 1).

13 clones of this transformation were checked, and four clones with the correct orientation (Nos. 13, 15, 16 and 18) were identifiable. Clone No. 16 was used further. The sequence of the contained construct (pcDNA3.1(V5-His) hp75 No. 16; cf. also FIG. 3d) is depicted in SEQ ID NO: 8. Further information on this sequence is summarized in the following section.

| | | |
|---|---|---|
| Human p75 | start: 40 | stop: 1320 |
| Neomycin | start: 2596 | stop: 3390 |
| Ampicillin | start: 4897 | stop: 5757 compl. |
| Bgh pA | start: 1487 | stop: 1701 |
| SV40 pA | start: 3416 | stop: 3654 |
| His tag | start: 1441 | stop: 1458 |
| V5 epitopes | start: 1397 | stop: 1438 |
| F1 ori | start: 1764 | stop: 2177 |
| SV40 promoter + ori | start: 2242 | stop: 2567 |
| pUC ori | start: 4086 | stop: 4759 |

b) Preparation of pIRES hp75

The plasmid pIRES (Clontech, Palo Alto, USA) was cut with Xba I and Not I (Roche Diagnostics) using a restriction mixture (40 μl; 1.5 h at 37° C.) comprising 5 μg of DNA, 2 μl of Xba I, 2 μl of Not I, 4 μl of 10× buffer H (Roche) and 32 μl of double-distilled water. The excised fragment was isolated in a conventional way from a DNA gel using the Qiagen gel extraction kit.

The plasmid pcDNA3.1 hp75, prepared as described above, was cut with Spe I and and Not I (Roche Diagnostics) using a restriction mixture (40 μl; 1.5 h at 37° C.) comprising 5 μg of DNA, 2 μl of Spe I, 2 μl of Not I, 4 μl of 10× buffer H (Roche), 32 μl of double-distilled water. The excised fragment was isolated in a conventional way from a DNA gel using the Qiagen gel extraction kit.

The restriction fragments prepared in this way were ligated at 16° C. overnight using the following mixture: 5 μl of hp75 construct, 2 μl of cut pIRES, 1.5 μl of T4-DNA ligase (Roche), 3 μl of 10× buffer T4 (Roche), 20.5 μl of double-distilled water. This ligation, which is referred to as pIRES hp75, was then transformed into the bacterial strain SuperComp XL2 Blue (Stratagene).

c) Preparation of pIRES hNgR hp75 (FIG. 3a)

pcDNA hNgR CDS1, obtained commercially from RZPD (Deutsches Resourcenzentrum für Genomforschung GmbH, clone No.: IRAL-p962L1427Q2), comprising the coding sequence for human Nogo receptor, was digested using a restriction mixture of 13 μl; 1 h at 37° C.) comprising 5 μg of DNA, 1.5 μl of Hind III (Roche), 1.5 μl of 10× buffer B (Roche) and 10 μl of double-distilled water (blunt end). The excised fragment was isolated in a conventional way from a DNA gel using the Qiagen gel extraction kit.

The ends were filled in by incubating 20 μl of the cut pcDNA h NgR CDS1 construct in a mixture (100 μl; 30 min 11° C.) comprising 2 μl of T4-DNA polymerase (Roche), 10 μl of 10× buffer T4 (Roche), 1 μl of 100 mM DTT (Gibco), 10 μl of 20 mM NTP mix (Stratagene) and 77 μl of double-distilled water. The T4 fill-in was isolated from a DNA gel using the Qiagen gel extraction kit.

For the dephosphorylation in order to avoid religation of the blunt ends, 50 μl of the T4 fill-in of the h NgR CDS1 construct were incubated with 2 μl of alkaline phosphatase (Roche), 10 μl of 10× buffer (Roche) and 38 μl of double-distilled water initially at 37° C. for 30 min and then at 65° C. for 15 min. The dephosphorylation construct obtained in this way was finally purified using the Qiagen Hit clean-up.

The purified pcDNA h NgR Hind III blunt fragment was then digested with EcoRI using a restriction mixture (15 μl, 1 h at 37° C.) comprising 10 μl of eluate (from Qiagen Hit clean-up), 1.5 μl of EcoRI (Roche), 1.5 μl of 10× buffer H (Roche) and 2.0 μl of double-distilled water and isolated after restriction from a DNA gel using the Qiagen gel extraction kit.

pIRES hp75 (prepared as above) was opened with EcoR I and Hind III using a restriction mixture (27 μl, 1.5 h at 37° C.) comprising 5 μg of DNA, 1.5 μl of EcoR I, 1.5 μl of Hind III, 4 μl of 10× buffer H (Roche) and 20 μl of double-distilled water. The desired hP75 fragment was isolated after restriction from a DNA gel using the Qiagen gel extraction kit and ligated with the hNgR construct prepared above, using a ligation mixture comprising 5 pNgR construct, 2 μl of cut pIRES hp75, 1.5 μl of T4-DNA ligase (Roche), 3 μl of 10× buffer T4 (Roche) and 20.5 μl of double-distilled water at 16° C. overnight. The resulting construct pIRES hNgR hp75 (FIG. 3a; SEQ ID NO: 10) was then transformed into the bacterial strain SuperComp XL2 Blue (Stratagene).

Further sequence information on pIRES hNgR hp75 is summarized in the following section:

| | |
|---|---|
| CMV prom. | 108-857 |
| NOGO R | 1202-2620 |
| IRES | 2658-3238 |
| hp75 | 3294-4574 |
| SV40pA | 4648-4869 |
| f1ori | 4964-5419 |
| Neo | 5483-6856 |
| amp | 7261-8121 |
| pUC ori | 8266-93 compl. |

d) Preparation of pcDNA4(mycHis)A hRhoA wt (FIG. 3c)

The plasmid pOTB7 RhoA (obtained commercially from RZPD, Deutsches Resourcenzentrum fücr Genomforschung GmbH, clone No.: IRAL-p962A174), which comprises the coding sequence for human RhoA GTPase, was digested using the following restriction mixture (25 μl, 1.5 h at 37° C.) comprising 5 μg of DNA, 1.5 μl of EcoR I, 1.5 μl of Xho I (Roche), 3 μl of 10× buffer H (Roche) and 19 μl of double-distilled water.

In a second mixture, pcDNA4(mycHis) (Invitrogen, Carlsbad, USA) was likewise digested using the following restriction mixture (25 μl, 1.5 h at 37° C.) comprising 5 μg of DNA, 1.5 μl of EcoR 1, 1.5 μl of Xho I (Roche), 3 μl of 10× buffer H (Roche) and 19 μl of double-distilled water.

The cut fragments were then purified and ligated as described above, resulting in the plasmid pcDNA4(mycHis)A h RhoA wt (cf. FIG. 3c: SEQ ID NO: 13). The resulting construct was then transformed into the bacterial strain Supercomp XL2 Blue (Stratagene).

Further sequence information on pcDNA4(mycHis)A hRhoA wt is summarized in the following section:

| | |
|---|---|
| CMV prom. | 197-851 |
| RhoA | 1058-1636 |
| His | 2437-2454 |
| myc | 2392-2421 |
| Bgh pA | 2480-2707 |
| SV40 pA | 4143-4273 |
| f1ori | 2753-3181 |
| amp | 5477-6334 compl. |
| pUC ori | 4656-5329 |
| Zeo | 3639-4010 |
| EM7 prom. | 3565-3620 |
| SV40 prom. | 3209-3517 |

d) Preparation of pcDNA3 hRhoA wt (FIG. 3b)

The preparation takes place in analogy to pcDNA4(mycHis)A hRhoA wt, but using the plasmid pcDNA3.1V5-His TOPO described above instead of pcDNA4(mycHis).

Further sequence information on pcDNA3 hRhoA wt (cf. FIG. 3b; SEQ ID NO: 15) is summarized in the following section:

| | |
|---|---|
| CMV prom. | 6124-6778 |
| h RhoA | 127-705 |
| Bgh pA | 1478-1718 |
| SV40 pA | 3402-3597 |
| SV40 ori | 2259-2584 |
| ColE1 ori | 4141-4515 |
| f1ori | 1788-2201 |
| Neo | 2620-3411 |
| amp | 4919-5779 compl. |

### Example 3

### Generation of Stable Recombinant HEK Cell Lines

a) Preparation of the Double Transfectant HEK293 NgR/p75

HEK293 wild-type cells (cultured in RPMI Glutamax+10% dial. FCS+1% antibiotic-antimycotic) were transfected in a first transfection step with the plasmid pIRES hNgR hp75. For this purpose for each mixture, 2 μg of plasmid DNA were mixed in 100 μl of serum-free RPMI-Gutamax medium and 2×10⁶ cells in 12 μl of LIPOFECTAMINE in 100 μl of serum-free RPMI-Gutamax medium in culture dishes with 10 wells, and incubated at room temperature for 15 to 20 minutes. The total volume was then made up to 1 ml for each transfection mixture using serum-free medium. Subsequently, 2 ml of serum-free RPMI-Gutamax medium were added to each dish, and incubation was carried out at 37° C. for 6 h. The medium was then changed to RPMI-Glutamax+5% dialyzed FCS and incubation was carried out at 37° C. for 1 day. The contents of the dishes were then split (in various dilutions: 1:10; 1:50, 1:100, 1:250, 1:500, 1:1000; 1 mixture/dish per dilution) (in RPMI-Glutamax+10% dialyzed FCS+1% antibiotic-antimycotic+G418; 800 μg/ml)

Clones were isolated from the dilution which yielded the first separated clones. Sterile glass minicylinders (BASF) were cautiously dipped by one end in sterile petrolatum (BASF) using sterile tweezers. The petrolatum-wetted end of the glass minicylinders was cautiously placed over the previously selected single clones. The single clone should be completely surrounded by the glass minicylinder. Then an Eppendorf pipette with a sterile tip was used to add 40 μl of trypsin (gibco, trypsin-EDTA) to the glass minicylinder. The trypsin was allowed to act on the cells for 1-2 minutes. The cells were resuspended by drawing up and discharging the trypsin with an Eppendorf pipette (sterile tip) several times (34 times). The resuspended cells were transferred with the Eppendorf pipette completely from the glass minicylinder into a 24-well plate (tissue culture plate, Falcon, Becton Dickinson, each well contained 1 ml of RPMI-Glutamax medium+5% dialyzed FCS).

Positive clones were detected by FACS as described above. Overexpression of the receptors NgR and p75 on the cell surface was moreover determined (results not shown).

b) Preparation of the Triple Transfectant HEK293 RhoA/NgR/p75

A positive clone prepared as in a) (clone 5) of the cell line HEK293 NgR/p75 is transfected in an analogous manner with the plasmid pcDNA4(mycHis)A hRhoA. In the last step, the mixtures are split in RPMI-Glutamax (+10% dialyzed FCS+1% antibiotic-antimycotic+G418; 800 μg/ml, +zeocin 125 μg/ml)

Positive clones were detected by testing them for RhoA expression by immunoblotting and for expression of the NgR and p75 receptors by FACS analysis.

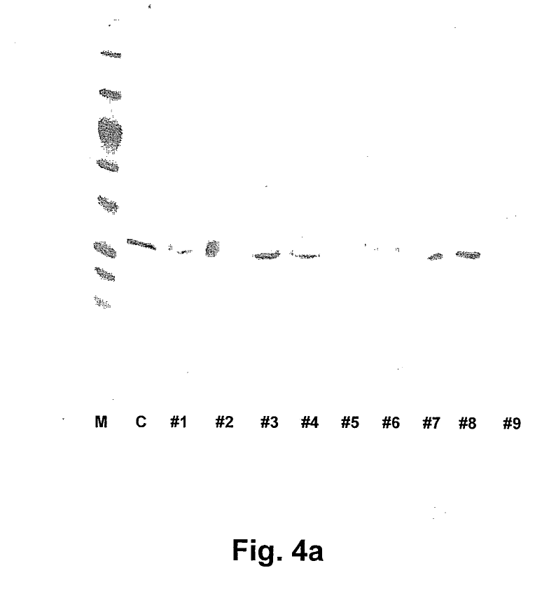

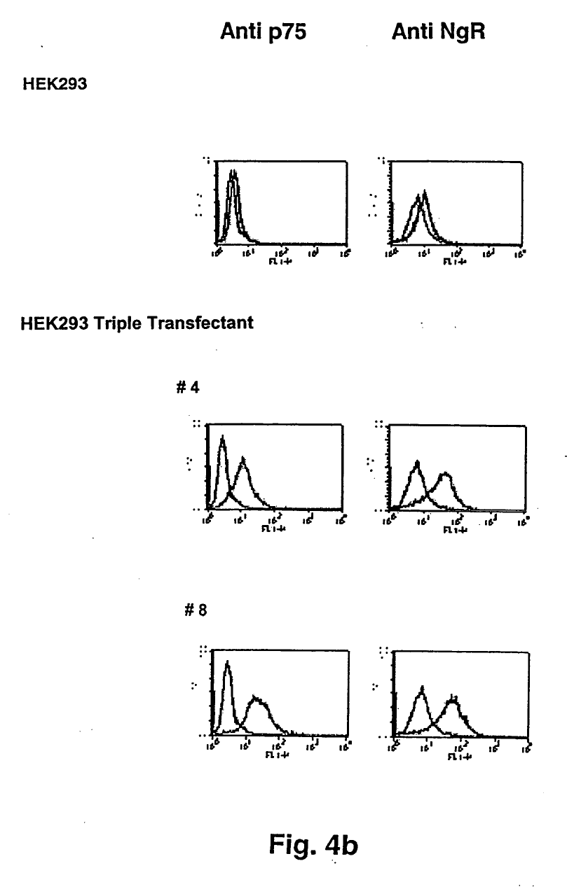

To detect RhoA, a cell homogenate derived from the particular clone was fractionated by SDS-PAGE gel electrophoresis (NuPAGE polyacrylamide gel 4-12%, 1.5 mm thick (Invitrogen Carlsbad, USA); the protein samples were denatured with 5% mercaptoethanol) and, after immunoblotting, tested with monoclonal mouse anti-RhoA antibody by a standard method. A typical result is depicted in FIG. 4a. A RhoA band with a molecular weight of about 21 kD is observed with positive clones.

Expression of NgR and p75 was detected by carrying out a FACS analysis as described above. A typical result is depicted in FIG. 4b.

c) Preparation of the Single Transfectant HEK293 RhoA

HEK293 wt cells are transfected with the plasmid pcDNA3 hRhoA wt. In the last step, the mixtures are split in RPMI-Glutamax (+10% dialyzed FCS+1% antibiotic-antimycotic+G418; 800 μg/ml). The procedure was otherwise analogous to a) and b).

### Example 4

### Stimulation of NgR with AP-Nogo-66 Activates Rho

a) RBD Assay

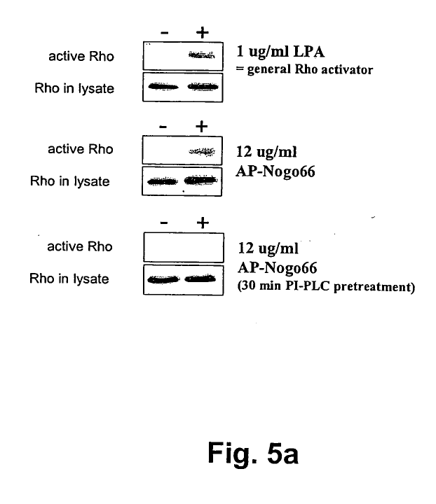

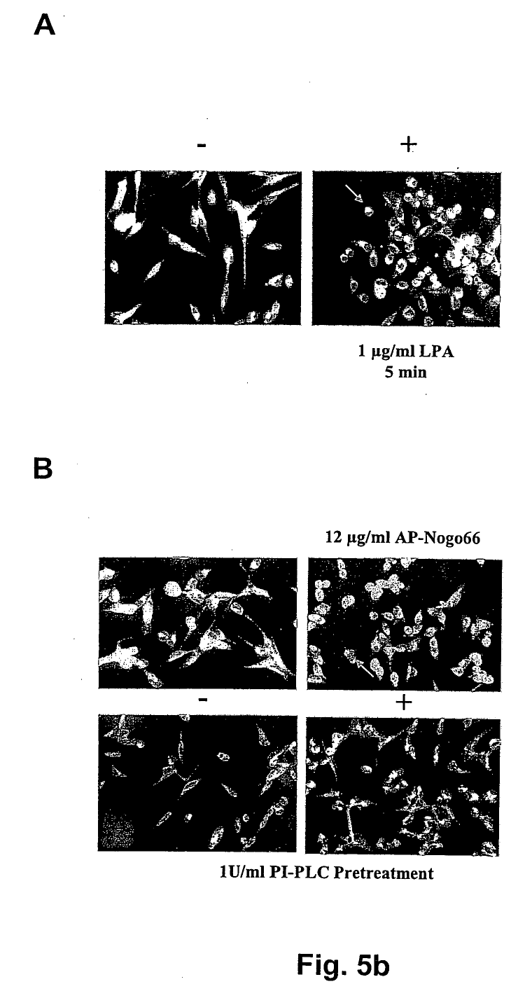

It was investigated, using the RBD assay described above, whether Rho is activated after stimulation of NgR with myelin proteins, such as Nogo-66, in HEK293-Rho cells which express NgR and p75 (HEK293-Rho/NgR/p75). The results are depicted in appended FIG. 5a.

Approach I: HEK293-RhoA/NgR/p75 cells were either not stimulated (−) or stimulated with L-alpha-lysophosphatidic acid (LPA) for 5 minutes (+). LPA is a general Rho activator which serves as positive control of the activity of GTPase in various cells. Stimulation with LPA led to a rapid activation of Rho in HEK293-RhoA/NgR/p75 cells and HEK wild-type cells (data not shown). This shows that the LPA-induced Rho activation is not dependent on NgR expression in the cell system of the invention.

Approach II: HEK293-RhoA/NgR/p75 cells were either not stimulated (−) or stimulated with AP-Nogo-66 for 5 minutes (+). Stimulation with Nogo-66 induced Rho activity with a similar time course compared with LPA treatment.

Approach III: HEK293-RhoA/NgR/p75 cells were treated with 1 U/ml PI-PLC 30 minutes before starting the experiment. The cells were then either not stimulated (−) or stimulated with AP-Nogo-66 (+) as described above. Stimulation with AP-Nogo-66 after release of NgR from the cell membrane led to no Rho activation. This illustrates that this GTPase is part of a signal cascade which is induced specifically via the ligand-activated NgR.

Each experiment was repeated at least 3 times.

b) ACR Assay

HEK293-Rho/NgR/p75 cells were transferred into microtiter plates with 96 wells (Biocoat Poly D-Lysine Black Clear Plates from Becton Dickinson, Heidelberg, order number 356640). After a starvation period (cf. above) of 2 hours, the cells were preincubated with PI-PLC (1 U/ml) for 30 minutes or not treated. The cells were subsequently stimulated with LPA or AP-Nogo-66 or remained unstimulated. Fluorescence micrograms were obtained as described above. Representative appearances are depicted in FIG. 5b.

The appearances shown in FIG. 5b demonstrate the surprising finding that a significant number of Rho-expressing HEK293-RhoA/NgR/p75 cells show changes in the cytoskeleton after stimulation, such as, for example, a rounding of the cells, accompanied by a cell shrinkage and the formation of dynamic cell projections. Examples of representative cells are identified by arrows in FIG. 5b.

As the above experiments demonstrate, these morphological changes depend on Rho activation and are not observable in unstimulated, receptor-competent cells (FIG. 5b II, left-hand appearance). These changes are also not observable in NgR-expressing cells after PI-PLC treatment and AP-Nogo-66 stimulation (FIG. 5b III, right-hand image).

### Example 5

### Image Analysis of Micrographs from ACR Assays of the Invention

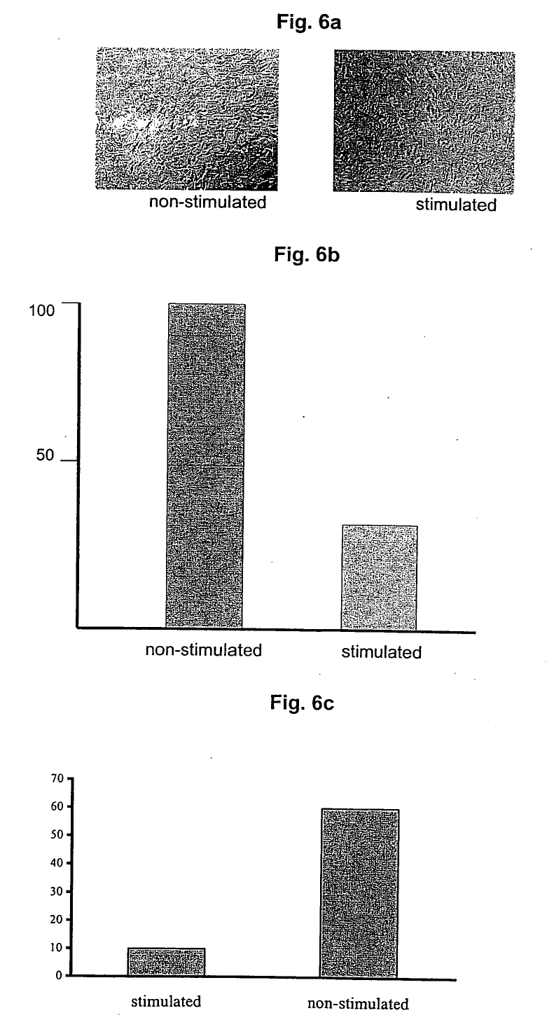

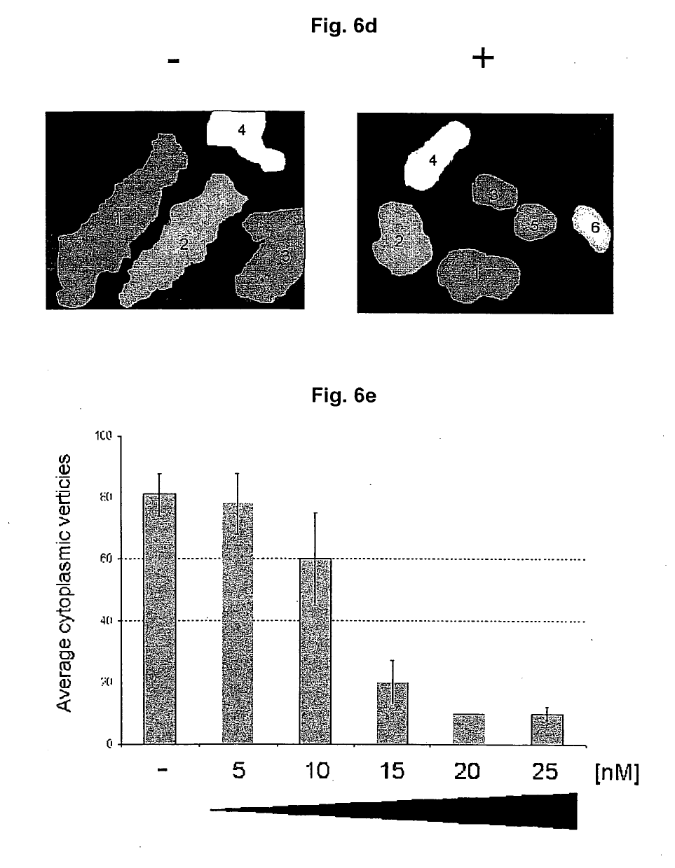

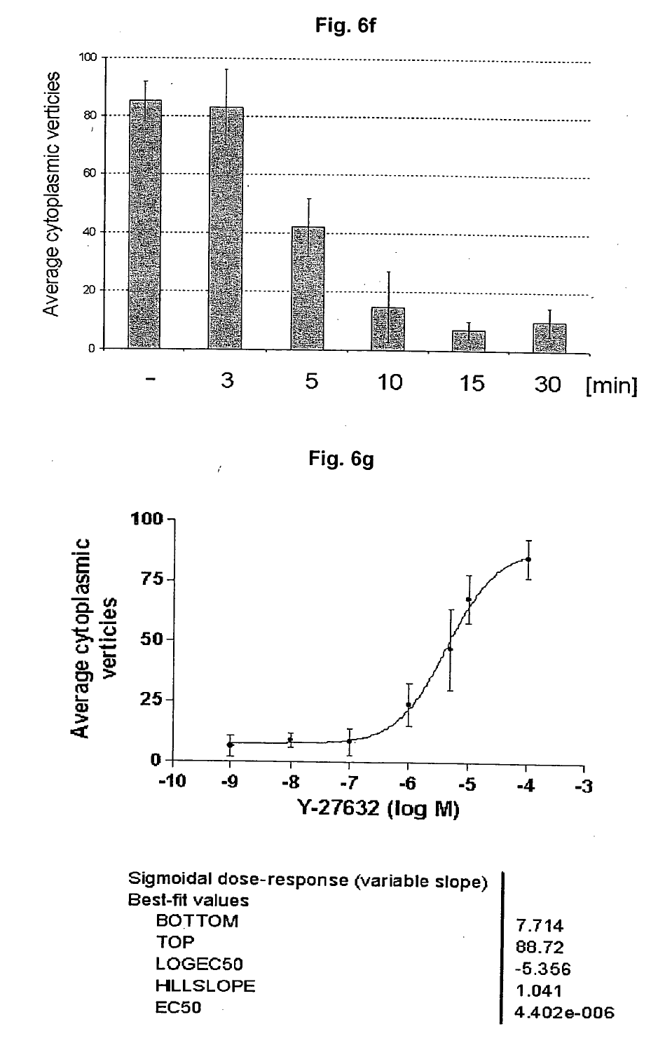

As illustrated by the attached FIGS. 6a to 6g an analysis of the data of the invention with image analysis techniques makes it possible to obtain rapid information about potential NgR antagonists. The use of high content image analysis screening systems makes it possible to avoid problems such as lack of reproducibility and long time requirement with which the conventional RBD assay is typically associated.

Fluorescence micrograms (FIG. 6a) obtained from an ACR assay carried out according to the invention (stimulation of HEK293-RhoA/NgR/p75 cells with 10 μg/ml AP-Nogo-66) were investigated with various high content screening systems, e.g. with the Atto Pathway HT image analysis system from BD Bioimages (Becton Dickinson) or the Discovery 1 from Molecular Devices, for changes in the cell morphology. FIG. 6b shows the result of the determination of the mean cell length in percent as determined by means of the Discovery 1 system. The Pathway HT was used to determine the number of so-called cytoplasmic vertices (cytoplasmic vertices are a measure of changes in the cytoskeleton which describes inter alia the contraction of actin filaments and the formation of stress fibers). The result of this evaluation is depicted in FIG. 6c. A significant, optically analyzable morphological change is observed in the cells of the invention after stimulation with the known NgR ligand.

A further specific example of a cell-based assay making use of a specific algorithm, which allows a robust and reproducible image analysis will now be described.

To analyze the reported morphological changes, 1×10⁴ cells per well were seeded into 96-well plates, fixed, the actin filaments were stained with FITC-labelled Alexa Phalloidin and the nuclei were visualized with DAPI. The plates were subjected to our high-content screening (HCS) system, the Pathway HT (Becton Dickinson) and the corresponding image analysis software Attovision. Analyzing the perimeter of the actin cytoskeleton achieved the most significant difference when comparing stimulated with unstimulated samples. Representative images demonstrating the segmentation and algorithm detection are shown in FIG. 6d. The perimeter of the actin cytoskeleton is calculated as “cytoplasmic verticies”, which is a measure for the number of turns or changes in direction that lines outlining the cell follow.

Using this parameter, the dose response dependency of AP-Nogo66 in HEK293-RhoA/NgR/p75 was investigated (FIG. 6e). The average number of cytoplasmic verticies of unstimulated cells, which served as a control, was determined and compared to the average number of cells stimulated with increasing concentrations of AP-Nogo66. Attovision analysis of the AP-Nogo66-induced cell contraction revealed a maximal reduction of the average perimeter by approximately 80-90% in comparison to untreated control cells after stimulation with 20 nM AP-Nogo66 (FIG. 6e). Each bar represents the mean value from at least three independent wells. The determined standard deviations demonstrate the significance and the robustness of the evaluated parameter difference.

Time dependency of AP-Nogo66-induced actin cytoskeletal rearrangement was also investigated (FIG. 6f. Again each bar represents the mean value from at least three independent wells. Stimulation of HEK293-RhoA/NgR/p75 cells with AP-Nogo66 led to rapid morphological changes within 5 min and reached a maximum after 15 min, a time course which confirms the Rho activation time course determined with the classical pulldown assay in the same cell line.

Due to the availability of different Rho over-expressing cell lines, signal transduction studies could be performed in a very controlled manner and were used for validation of the actin cytoskeletal rearrangment (ACR) assay. Activated GTP-bound Rho is able to recruit and activate the serine-threonine kinase ROCK, a specific downstream target of Rho. ROCK activation leads to phosphorylation of several cytoskeletal proteins, resulting in cell contraction (Mills et al., 1998, J. Cell Biol 140(3):627-636). Inhibition of ROCK with Y-27632 (purchased from Calbiochem, La Jolla, Calif., USA) abolished AP-Nogo66 induced Rho-dependent cell rounding and shrinkage in a dose-dependent manner (FIG. 6g), confirming that NgR induced cell morphology changes requires the activity of this particular effector protein linking Rho activation directly to the actin cytoskeleton. Furthermore, pharmacological analysis of the inhibitory effect of Y-27632 on the actin cytoskeletal rearrangement underlined good reproducibility. The determined IC50 value for Y-27632 in this assay set-up ranges in the order of magnitude of what had described for this broadly used compound (Mills et al., 1998). Moreover, determination of a Z′ factor of 0.6 (n=30 wells analyzed) demonstrates good robustness of the described assay format for compound testing.

1. A recombinant cell line which expresses:
a) a functional intracellular factor which is involved in a signal pathway which induces a change in the cytoskeleton of the cell; and/or
b) a membrane-bound receptor which interacts with the intracellular factor.

2. The cell line according to claim 1, where the intracellular factor is a Rho GTPase, and/or the membrane-bound receptor comprises a functional neutrotrophin receptor p75 and a functional Nogo receptor (NgR).

3. The cell line according to claim 2, where
a) the functional RhoGTPase comprises an amino acid sequence as shown in SEQ ID NO: 16;
b) the functional p75 comprises an amino acid sequence as shown in SEQ ID NO: 9; and
c) the functional NgR comprises an amino acid sequence as shown in SEQ ID NO: 11.

4. The cell line according to claim 1, where cells are able to reorganize their actin cytoskeleton induced by the binding of a ligand to NgR.

5. The cell line according to claim 4, where the ligand binding induces an activation of Rho.

6. The cell line according to claim 4, where the ligand comprises Nogo-A or an NgR-binding fragment or derivative derived therefrom.

7. The cell line according to claim 1, derived from an adherent mammalian cell line with extended cell morphology.

8. The cell line according to claim 7, derived from a human epithelial or fibroblast cell line.

9. The cell line according to claim 8, derived from a human embryonic kidney (HEK) cell line.

10. The use of a cell line according to claim 1 for determining effectors of NgR-dependent signal transduction.

11. An assay method for determining changes in the cytoskeleton of a cell line to be investigated, where a recombinant cell line as defined in claim 1, or a corresponding primary cell line is cultivated with a ligand which interacts with the cells and which, if appropriate, brings about the change in the cytoskeleton via induction of an intracellular signal pathway, and changes in the reorganization of the cytoskeleton of the cell lines treated in this way are determined.

12. The assay method according to claim 11, where the ligand interacts with a membrane-bound receptor.

13. The assay method according to claim 11, where the cell line is culivated in the presence or absence of an analyte which is suspected to comprise an effector which modulates the effect of the ligand on the cell.

14. The assay method according to claim 13, where the cultivation with the analyte takes place simultaneously or sequentially in relation to the cultivation with the receptor-binding ligand.

15. The assay method according to claim 11 for determining effectors of NgR-dependent activation of the GTPase Rho, where the recombinant cell line or a corresponding primary cell line is cultivated in the presence of an NgR-binding ligand and in the presence or absence of an analyte suspected to comprise the effector, and differences in the reorganization of the cytoskeleton of the cell lines treated in this way are determined.

16. The assay method according to claim 11, where the average of at least one of the following parameters is determined for a representative cell population:
a) cell circumference
b) cell length
c) cell width
d) cell area
e) length of the cell projections
f) cell density
g) cytoplasmic area
h) cytoplasm vertices
i) refractive index
j) cell-free area
k) area covered with cells
l) a mathematical linkage of at least two of parameters a) to k), or a parameter which can be derived from at least one of the parameters a) to k), such as, in particular, the ratio of cell length to cell width, or the ratio of cell circumference squared to cell area; or
m) an appropriate reciprocal value of one of the parameters a) to 1).

17. The assay method according to claim 11, where the determination takes place by means of an automated method such as, in particular, by means of an automatic image analysis system.

18. The use of an assay method according to claim 11 for determining effectors of neuronal regeneration.

19. The use according to claim 18, where the effector partly or completely inhibits Rho activation.

20. The use according to claim 18, where the effector promotes neuronal regeneration.

21. The use according to claim 10, where the effector partly or completely neutralizes a disease- or injury-related inhibition of axonal regeneration or a disease- or injury-related collapse of the neuronal growth cone.

22. The use according to claim 18, where the effector is an NgR antagonist.

23. The use according to claim 18 in a screening method, in particular a high content screening method for effectors of neuronal regeneration.

24. A vector comprising a nucleic acid sequence coding for functional RhoGTPase or comprising nucleic acid sequences coding for functional p75 and functional NgR.

25. A method for producing a recombinant cell line according to claim 1, where a wild-type cell line is transformed with at least one coding nucleic acid sequence which codes for a functional RhoGTPase and, if appropriate, for p75 and NgR.

26. The method according to claim 25, where the cell line is transformed with at least one vector comprising a nucleic acid sequence coding for functional RhoGTPase or comprising nucleic acid sequences coding for functional p75 and functional NgR.

27. A high content screening method for determining changes in the cytoskeleton of a cell line to be investigated and in particular for determining effectors of NgR-dependent activation of the GTPase Rho, where an assay method according to claim 11 is used.

28. An apparatus for carrying out a method according to claim 27.

29. A test kit comprising a recombinant cell line according to claim 1 or a corresponding primary cell line, and a ligand for the cellular receptor.


SEQUENCE LISTING (16 sequences)

SEQ ID NO: 1 - DNA, 31 bp, Artificial Sequence
  PCR primer mez402
gccaccatgaggatatacaagggtgtgatcc

SEQ ID NO: 2 - DNA, 24 bp, Artificial Sequence
  PCR primer mez404
cttcagagaatcaactaaatcatc

SEQ ID NO: 3 - DNA, 20 bp, Artificial Sequence
  PCR primer mey35
tcacactggggatgtggcag

SEQ ID NO: 4 - DNA, 24 bp, Artificial Sequence
  PCR primer mey36
gccaccatgggggcaggtgccacc

SEQ ID NO: 5 - DNA, 18 bp, Artificial Sequence
  PCR primer mey71
gcagccccatcagtccgc

SEQ ID NO: 6 - DNA, 5523 bp, Artificial Sequence
  Plasmid pcDNA3.1V5-HisTOPO
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttggtaccgagctcggatccactagtccagtgtggtggaattgcccttaagggcaattctgcagatatccagcacagtggcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 7 - DNA, 6850 bp, Artificial Sequence
  Plasmid pAP-tag/PPC/hNOGO66 Nr.5
aattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccaccatggagacagacacactcctgctatgggtactgctgctctgggttccaggttccactggtgacgcggcccagccggccaggcgcgcgcgccgtacgtacgaagcttacgtaagatcttccggaatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcccatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttatctcgaggaagcgctctctctagatctggaagttctgttccaggggcccctgggatctaggatatacaagggtgtgatccaagctatccagaaatcagatgaaggccacccattcagggcatatctggaatctgaagttgctatatctgaggagttggttcagaagtacagtaattctgctcttggtcatgtgaactgcacgataaaggaactcaggcgcctcttcttagttgatgatttagttgattctctgaagtagtctagaagggcccgaacaaaaactcatctcagaagaggatctgaatagcgccgtcgaccatcatcatcatcatcattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgac

SEQ ID NO: 8 - DNA, 13612 bp, Artificial Sequence
  Plasmid pcDNA3.1(V5-His)hp75 Nr.16
gatccactagtccagtgtggtggaattgcccttgccaccatgggggcaggtgccaccggccgcgccatggacgggccgcgcctgctgctgttgctgcttctgggggtgtcccttggaggtgccaaggaggcatgccccacaggcctgtacacgcacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtggaacagctgcaagcagaacaagcaaggagccaacagccggccagtgaaccagacgcccccaccagagggagaaaaactccacagcgacagtggcatctccgtggacagccagagcctgcatgaccagcagccccacacgcagacagcctcgggccaggccctcaagggtgacggaggcctctacagcagcctgcccccagccaagcgggaggaggtggagaagcttctcaacggctctgcgggggacacctggcggcacctggcgggcgagctgggctaccagcccgagcacatagactcctttacccatgaggcctgccccgttcgcgccctgcttgcaagctgggccacccaggacagcgccacactggacgccctcctggccgccctgcgccgcatccagcgagccgacctcgtggagagtctgtgcagtgagtccactgccacatccccagtgtgaaagggcaattctgcagatatccagcacagtggcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtccgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttggtaccgagctcggatccactagtccagtgtggtggaattgcccttgccaccatgggggcaggtgccaccggccgcgccatggacgggccgcgcctgctgctgttgctgcttctgggggtgtcccttggaggtgccaaggaggcatgccccacaggcctgtacacgcacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtggaacagctgcaagcagaacaagcaaggagccaacagccggccagtgaaccagacgcccccaccagagggagaaaaactccacagcgacagtggcatctccgtggacagccagagcctgcatgaccagcagccccacacgcagacagcctcgggccaggccctcaagggtgacggaggcctctacagcagcctgcccccagccaagcgggaggaggtggagaagcttctcaacggctctgcgggggacacctggcggcacctggcgggcgagctgggctaccagcccgagcacatagactcctttacccatgaggcctgccccgttcgcgccctgcttgcaagctgggccacccaggacagcgccacactggacgccctcctggccgccctgcgccgcatccagcgagccgacctcgtggagagtctgtgcagtgagtccactgccacatccccagtgtgaaagggcaattctgcagatatccagcacagtggcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtccgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttggtaccgagctcg

SEQ ID NO: 9 - protein, 427 aa, Artificial Sequence
  Plasmid pcDNA3.1(V5-His)hp75 Nr.16
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSCKQNKQGANSRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

SEQ ID NO: 10 - DNA, 8846 bp, Artificial Sequence
  Plasmid pIRES hNgR hp75
gtgatgctcgtcaggggggcggagcctatggaaaaacgccagcaacgcggcctttttacggttcctggccttttgctggccttttgctcacatggctcgacagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagagcttatgaagagggcgtccgctggagggagccggctgctggcatgggtgctgtggctgcaggcctggcaggtggcagccccatgcccaggtgcctgcgtatgctacaatgagcccaaggtgacgacaagctgcccccagcagggcctgcaggctgtgcccgtgggcatccctgctgccagccagcgcatcttcctgcacggcaaccgcatctcgcatgtgccagctgccagcttccgtgcctgccgcaacctcaccatcctgtggctgcactcgaatgtgctggcccgaattgatgcggctgccttcactggcctggccctcctggagcagctggacctcagcgataatgcacagctccggtctgtggaccctgccacattccacggcctgggccgcctacacacgctgcacctggaccgctgcggcctgcaggagctgggcccggggctgttccgcggcctggctgccctgcagtacctctacctgcaggacaacgcgctgcaggcactgcctgatgacaccttccgcgacctgggcaacctcacacacctcttcctgcacggcaaccgcatctccagcgtgcccgagcgcgccttccgtgggctgcacagcctcgaccgtctcctactgcaccagaaccgcgtggcccatgtgcacccgcatgccttccgtgaccttggccgcctcatgacactctatctgtttgccaacaatctatcagcgctgcccactgaggccctggcccccctgcgtgccctgcagtacctgaggctcaacgacaacccctgggtgtgtgactgccgggcacgcccactctgggcctggctgcagaagttccgcggctcctcctccgaggtgccctgcagcctcccgcaacgcctggctggccgtgacctcaaacgcctagctgccaatgacctgcagggctgcgctgtggccaccggcccttaccatcccatctggaccggcagggccaccgatgaggagccgctggggcttcccaagtgctgccagccagatgccgctgacaaggcctcagtactggagcctggaagaccagcttcggcaggcaatgcgctgaagggacgcgtgccgcccggtgacagcccgccgggcaacggctctggcccacggcacatcaatgactcaccctttgggactctgcctggctctgctgagcccccgctcactgcagtgcggcccgagggctccgagccaccagggttccccacctcgggccctcgccggaggccaggctgttcacgcaagaaccgcacccgcagccactgccgtctgggccaggcaggcagcgggggtggcgggactggtgactcagaaggctcaggtgccctacccagcctcacctgcagcctcacccccctgggcctggcgctggtgctgtggacagtgcttgggccctgctgagaattcacgcgtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagtccagtgtggtggaattgcccttgccaccatgggggcaggtgccaccggccgcgccatggacgggccgcgcctgctgctgttgctgcttctgggggtgtcccttggaggtgccaaggaggcatgccccacaggcctgtacacgcacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtggaacagctgcaagcagaacaagcaaggagccaacagccggccagtgaaccagacgcccccaccagagggagaaaaactccacagcgacagtggcatctccgtggacagccagagcctgcatgaccagcagccccacacgcagacagcctcgggccaggccctcaagggtgacggaggcctctacagcagcctgcccccagccaagcgggaggaggtggagaagcttctcaacggctctgcgggggacacctggcggcacctggcgggcgagctgggctaccagcccgagcacatagactcctttacccatgaggcctgccccgttcgcgccctgcttgcaagctgggccacccaggacagcgccacactggacgccctcctggccgccctgcgccgcatccagcgagccgacctcgtggagagtctgtgcagtgagtccactgccacatccccagtgtgaaagggcaattctgcagatatccagcacagtggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgattttt

SEQ ID NO: 11 - protein, 473 aa, Artificial Sequence
  Plasmid pIRES hNgR hp75
MKRASAGGSRLLAWVLWLQAWQVAAPCPGACVCYNEPKVTTSCPQQGLQAVPVGIPAASQRIFLHGNRISHVPAASFRACRNLTILWLHSNVLARIDAAAFTGLALLEQLDLSDNAQLRSVDPATFHGLGRLHTLHLDRCGLQELGPGLFRGLAALQYLYLQDNALQALPDDTFRDLGNLTHLFLHGNRISSVPERAFRGLHSLDRLLLHQNRVAHVHPHAFRDLGRLMTLYLFANNLSALPTEALAPLRALQYLRLNDNPWVCDCRARPLWAWLQKFRGSSSEVPCSLPQRLAGRDLKRLAANDLQGCAVATGPYHPIWTGRATDEEPLGLPKCCQPDAADKASVLEPGRPASAGNALKGRVPPGDSPPGNGSGPRHINDSPFGTLPGSAEPPLTAVRPEGSEPPGFPTSGPRRRPGCSRKNRTRSHCRLGQAGSGGGGTGDSEGSGALPSLTCSLTPLGLALVLWTVLGPC

SEQ ID NO: 12 - protein, 427 aa, Artificial Sequence; identical to SEQ ID NO: 9
  Plasmid pIRES hNgR hp75

SEQ ID NO: 13 - DNA, 6482 bp, Artificial Sequence
  Plasmid pcDNA4(mycHis)A hRhoA wt
gatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttggtaccgagctcggatccactagtccagtgtggtggaattcggcacgaggctcgttagtccacggtctggtcttcagctacccgccttcgtctccgagtttgcgactcgcggaccggcgtccccggcgcgaagaggctggactcggattcgttgcctgagcaatggctgccatccggaagaaactggtgattgttggtgatggagcctgtggaaagacatgcttgctcatagtcttcagcaaggaccagttcccagaggtgtatgtgcccacagtgtttgagaactatgtggcagatatcgaggtggatggaaagcaggtagagttggctttgtgggacacagctgggcaggaagattatgatcgcctgaggcccctctcctacccagataccgatgttatactgatgtgtttttccatcgacagccctgatagtttagaaaacatcccagaaaagtggaccccagaagtcaagcatttctgtcccaacgtgcccatcatcctggttgggaataagaaggatcttcggaatgatgagcacacaaggcgggagctagccaagatgaagcaggagccggtgaaacctgaagaaggcagagatatggcaaacaggattggcgcttttgggtacatggagtgttcagcaaagaccaaagatggagtgagagaggtttttgaaatggctacgagagctgctctgcaagctagacgtgggaagaaaaaatctgggtgccttgtcttgtgaaaccttgctgcaagcacagcccttatgcggttaattttgaagtgctgtttattaatcttagtgtatgattactggcctttttcatttatctataatttacctaagattacaaatcagaagtcatcttgctaccagtatttagaagccaactatgattattaacgatgtccaacccgtctggcccaccagggtccttttgacactgctctaacagccctcctctgcactcccacctgacacaccaggcgctaattcaaggaatttcttaacttcttgcttctttctagaaagagaaacagttggtaacttttgtgaattaggctgtaactactttataactaacatgtcctgcctattatctgtcagctgcaaggtactctggtgagtcaccacttcagggctttactccgtaacagattttgttggcatagctctggggtgggcagttttttgaaaatgggctcaaccagaaaagcccaagttcatgcagctgtggcagagttacagttctgtggtttcatgttagttaccttatagttactgtgtaattagtgccacttaatgtatgttaccaaaaataaatatatctaccccagactagatgtagtattttttgtataattggatttcctaatactgtcatcctcaaagaaagtgtattggttttttaaaaaagaaagtgtatttggaaataaagtcagatggaaaattcaaaaaaaaaaaaaaaaaaactcgagtctagagggcccttcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcggga

SEQ ID NO: 14 - protein, 193 aa, Artificial Sequence
  Plasmid pcDNA4(mycHis)A hRhoA wt
MAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 15 - DNA, 6853 bp, Artificial Sequence
  Plasmid pcDNA3 hRhoA wt
aattcggcacgaggctcgttagtccacggtctggtcttcagctacccgccttcgtctccgagtttgcgactcgcggaccggcgtccccggcgcgaagaggctggactcggattcgttgcctgagcaatggctgccatccggaagaaactggtgattgttggtgatggagcctgtggaaagacatgcttgctcatagtcttcagcaaggaccagttcccagaggtgtatgtgcccacagtgtttgagaactatgtggcagatatcgaggtggatggaaagcaggtagagttggctttgtgggacacagctgggcaggaagattatgatcgcctgaggcccctctcctacccagataccgatgttatactgatgtgtttttccatcgacagccctgatagtttagaaaacatcccagaaaagtggaccccagaagtcaagcatttctgtcccaacgtgcccatcatcctggttgggaataagaaggatcttcggaatgatgagcacacaaggcgggagctagccaagatgaagcaggagccggtgaaacctgaagaaggcagagatatggcaaacaggattggcgcttttgggtacatggagtgttcagcaaagaccaaagatggagtgagagaggtttttgaaatggctacgagagctgctctgcaagctagacgtgggaagaaaaaatctgggtgccttgtcttgtgaaaccttgctgcaagcacagcccttatgcggttaattttgaagtgctgtttattaatcttagtgtatgattactggcctttttcatttatctataatttacctaagattacaaatcagaagtcatcttgctaccagtatttagaagccaactatgattattaacgatgtccaacccgtctggcccaccagggtccttttgacactgctctaacagccctcctctgcactcccacctgacacaccaggcgctaattcaaggaatttcttaacttcttgcttctttctagaaagagaaacagttggtaacttttgtgaattaggctgtaactactttataactaacatgtcctgcctattatctgtcagctgcaaggtactctggtgagtcaccacttcagggctttactccgtaacagattttgttggcatagctctggggtgggcagttttttgaaaatgggctcaaccagaaaagcccaagttcatgcagctgtggcagagttacagttctgtggtttcatgttagttaccttatagttactgtgtaattagtgccacttaatgtatgttaccaaaaataaatatatctaccccagactagatgtagtattttttgtataattggatttcctaatactgtcatcctcaaagaaagtgtattggttttttaaaaaagaaagtgtatttggaaataaagtcagatggaaaattcaaaaaaaaaaaaaaaaaaactcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccactagtaacggccgccagtgtgctgg

SEQ ID NO: 16 - protein, 193 aa, Artificial Sequence; identical to SEQ ID NO: 14
  Plasmid pcDNA3 hRhoA wt